(19) United States
(12) Patent Application Publication
(10) Pub. No.: US 2025/0019634 A1
(43) Pub. Date: Jan. 16, 2025

(54) LABORATORY SHAKERS

(71) Applicant: Eppendorf SE, Hamburg (DE)

(72) Inventors: Arne Schafrinski, Oldesloe (DE); Jan Fitzer, Ammersbek (DE)

(21) Appl. No.: 18/711,859

(22) PCT Filed: Nov. 24, 2022
(86) PCT No.: PCT/EP2022/083166

§ 371 Date: May 20, 2024

(30) Foreign Application Priority Data
Nov. 24, 2021 (EP) 21210317.0

(51) Int. Cl. C12M 3/06; C12M 1/00; C12M 3/00

(52) U.S. Cl. CPC C12M 27/16; C12M 23/46; C12M 41/14

(57) ABSTRACT

The invention relates to laboratory shakers for shaking laboratory samples stored in sample vessels in a chamber which comprises an pull-out mechanism which is extendable through a chamber opening for extending a sample platform storing the sample vessels in the extension direction A, which is shakable in a retracted position P1 and which preferably comprises a securing mechanism for securing the position P1, with a pivotable handle which is locked in a secured position S1 and whose unlocking and a pivoting movement beginning in the pull-out direction A releases the securing effected by the securing mechanism.

The invention relates to laboratory shakers for shaking laboratory samples stored in sample vessels, in particular microorganisms in suspension.

Temperature-controlled laboratory shakers are used in biological, medical and pharmaceutical laboratories for the cultivation of bacteria, yeast and other organisms in suspension. They are essential, for example, for the production of recombinant DNA, protein expression or the screening of cultures. As a laboratory shaker is primarily a shared piece of equipment that runs around the clock at high speeds and variable loads, it must be durable and reliable.

The parameters relevant to a user for a laboratory shaker, especially an incubation shaker, are first of all a certain target temperature in the sample storage room, a certain speed and a certain load-bearing capacity of the shaker platform. Most applications require the use of different vessel sizes and types: from plates for initial screening, to conical vessels for precultures, to large flasks for plasmid production or protein expression. The constant need for higher product yields has led to the invention of new flask types that offer better aeration than standard shake flasks. This allows the typical filling volume to be increased by up to 40%, resulting in a higher weight on the platform. Higher speeds above 250 rpm are common for applications with e.g. E. coli to achieve a significant increase in cell density. The requirements for laboratory shakers are therefore extensive and, in addition to continuous load capacity, include sufficient versatility to cope with all types of platform configurations, loads and also high speeds. Durability and robustness are required for reliable operation over many years.

Orbital shakers are laboratory shakers that move a platform so that s all points on the top of the platform move in the X-Y plane on a circular path with a common radius, as described, for example, in EP2714253B1. Generally, beakers, flasks and other vessels are attached to the top of the platform so that the liquid contained therein is swirled around the inner side walls of the vessel to increase mixing and enhance interaction or exchange between the liquid and the local gaseous environment.

Such laboratory shakers have a chamber for holding the laboratory samples to be tempered; this chamber is usually arranged inside a housing. Access to the chamber, where the user stores and removes the samples inside the housing, in particular in the chamber, is usually via a housing opening which can be closed by means of a housing door. In further embodiments, the chamber also comprises a gas supply. These types of devices allow cells to be cultivated in a CO₂ atmosphere. They are called incubation shakers.

A well-known laboratory shaker is the Innova® S44i, available from Eppendorf SE, Hamburg, Germany. It comprises a pull-out mechanism that allows a sample platform to be pulled out of the chamber, making loading and unloading the laboratory shaker much easier. The pull-out mechanism comprises a handle with a release button, which is locked in a vertical home position. To unlock, the handle is first moved against the pull-out direction towards the chamber when the unlock button is pressed, releasing the locking catch. The handle is then pulled away from the chamber while the locking button is pressed and brought downwards into a horizontal position in which the sample platform can be pulled outwards using the handle and the pull-out mechanism. This securing mechanism provides a high level of safety for securing the home position of the sample platform, in which it is fully positioned in the chamber and in which the shaking motion of the laboratory shaker is performed. However, the unlocking process was sometimes perceived by users as very complex and not very intuitive. In addition, the chamber of said laboratory shaker must be prepared in several steps for the purpose of cleaning or sterilization; in particular, the pull-out mechanism must be disassembled using tools and sufficient expertise regarding these steps. The chamber must be cleaned in particular if culture fluid (cell culture medium) has leaked, e.g. due to breakage of a sample vessel. In such situations, it is essential to clear the chamber as quickly as possible, in particular to remove the platform so that the chamber can be cleaned. The chamber must then be refilled and made ready for use. Here, too, speed is important in order to resume sample movement or cell cultivation without long delays. In the case of immobile CO₂ incubators, it is a well-known procedure to additionally disinfect the chamber with a hot temperature treatment of 180° C. for a certain number of hours.

The present invention is therefore based on the task of providing an improved laboratory shaker in which the securing mechanism for securing the basic position of the sample platform is, on the one hand, secure and, on the other hand, can be unlocked easily and intuitively, and in particular can be operated ergonomically with a single hand without having to apply major forces. The present invention is based on the further task of providing an improved laboratory shaker whose chamber is easier to clean.

The invention solves this problem in each case by means of the laboratory shaker according to claims 1, 16 and 19. Further technical solutions and preferred embodiments are mentioned in the description, and further preferred embodiments are also the subject of the subclaims.

The securing mechanism of a laboratory shaker according to the invention can be unlocked with one hand and by simultaneously actuating the actuating element and pulling the handle in pull-out direction A. This results in a flowing sequence of movements that is perceived by users as very ergonomic and intuitive. To unlock the securing mechanism, the-in particular spring-loaded-actuating element only needs to be pressed with the fingers of one hand, which causes the locking device, in particular the locking element and the locking opening on the carrier element, to be unlocked via a pulling mechanism. At the same time, the user pulls on the handle in pull-out direction A so that the handle moves downwards; the spring-loaded actuating element can be released again immediately, which is convenient and intuitive, the handle engages in the second handle position S2, in particular automatically and due to the spring tension, and the user pulls out the carrier device (including sample platform and vessels) with the same pull-out movement with which he also locked the handle. Securing in the opposite direction to the pull-out direction is similarly simple and intuitive. The forces to be applied by the user are low (see FIG. 9). This represents a significant simplification for the user, as it allows him to perform other tasks with the other hand during the smooth one-handed opening and closing of the laboratory shaker with a reduced risk of motor errors, and in particular to concentrate on these tasks. For example, it is typical for the user to carry sample vessels with a total mass of several kilograms that are to be placed in the laboratory shaker or that have been removed from it with the other hand while operating the laboratory shaker with one hand.

A laboratory shaker according to the invention for shaking samples stored in sample vessels has:
a temperature-adjustable chamber (2) with a closable chamber opening (3),
a carrier device (12) for carrying a sample platform (14) on which the sample vessels can be placed,
a pull-out mechanism (10) for pulling out the carrier device (12), which is arranged completely in the chamber in a first position (P1), which passes through the chamber opening (3) along a pull-out direction (A) during manual pull-out and which is arranged outside the chamber (2) in a second position (P2),
a shaking device for shaking the samples,
a securing mechanism (20) for securing and releasing the first position (P1), with a locking device (30) and a handle (40), which is fastened to the carrier device (12) and is movable between a first handle position (S1), in which the locking device (30) is locked, and a second handle position (S2), in which the locking device (30) is unlocked,
wherein a displacement of the first handle position (S1) can be blocked by the locking device (30) and the locking device (30) comprises a manually operable actuating element (42) arranged on the handle (40), by the actuation of which the locking device (30) can be unlocked in the first handle position (S1), so that the handle (40) can be moved into the second handle position (S2),
and wherein the securing mechanism (20) can be unlocked by simultaneously actuating the actuating element (42) and pulling the handle (40) in the pull-out direction (A).

The carrier device is used to support a sample platform on which the sample vessels can be placed. The carrier device is preferably a structural component which is attached in particular to the pull-out mechanism and which is arranged completely in the chamber in the first position (P1), which passes through the chamber opening during manual pull-out along the pull-out direction (A) and which is arranged outside the chamber in the second position (P2). The carrier device can have at least one, preferably more than one carrier element-preferably two carrier elements-each of which is preferably attached to a rail element of the pull-out mechanism. One or more, in particular two, rail elements of the pullout mechanism can serve as carrier elements of the carrier device. A carrier element can have an L-shaped profile, which is fastened in particular to a rail element. Two support elements can be connected by at least one connecting strut, in particular by a front support element, in particular a front profile. In particular, a base section of the handle can be provided on the front support element, in particular integrally formed or attached. The base section of the handle carries the handle in particular, in particular the pivot axis (X1) of the handle is formed on the base section of the handle.

Preferably, the carrier device comprises at least one, in particular at least two or three, positioning elements, in particular positioning pins, which are in particular firmly connected to the carrier device. The positioning elements serve to align, position and in particular fix the sample platform at least in one direction (x, y, z), preferably in the two directions x, y, of a Cartesian coordinate system in which the pull-out direction A extends parallel to the y-axis. In the direction of the negative z-axis, the sample platform rests in its inserted position on at least one support section of the carrier device, in particular of the at least one carrier element. In the direction of the positive z-axis, the sample platform can preferably be removed without tools.

Preferably, the pull-out mechanism, in particular the carrier device, comprises at least one sub-platform, in particular a plate element extending in an x-y plane, which can in particular be fastened between two carrier elements of the carrier device or which in particular forms a carrier element. A sub-platform can be set up to carry at least one positioning element and/or to support or carry the sample platform, in particular in the first position. However, the sample platform can also be firmly connected to the carrier device, in particular non-detachable or non-tool-free detachable.

In particular, the pull-out mechanism comprises or consists of a rail system. The rail system can have at least one rail element configured as a base rail element, which is set up for mounting, in particular fastening, in the chamber of the laboratory shaker, in particular for indirect or direct mounting/fastening to at least one connecting element of a shaking device arranged in the chamber. A sub-platform can serve as a connecting element. Base rail elements are in particular not configured to be pulled out of the chamber-the telescopic rail elements are used for this purpose. Preferably, the rail system comprises at least one rail element set up as a telescopic rail element, which is configured to slide or roll along the at least one base rail element along the pull-out direction A.

Preferably, the rail system comprises two base rail elements which are arranged or mounted close to opposite chamber walls in the chamber. Preferably, the rail system comprises two telescopic rail elements, whereby one telescopic rail element is arranged on a base rail element to perform a translational movement along the extension direction and, in particular, is connected to it. This allows a full extension to be realized, in which the sample platform is essentially fully extended out of the chamber in a second position (P2), resulting in convenient operation of the sample platform. In particular, a cascading rail system can be implemented in which at least one further telescopic rail element is arranged on at least one telescopic rail element in a sliding/rolling manner. For example, two further telescopic rail elements can be provided, one of which is arranged to slide/roll on one of the first-mentioned telescopic rail elements. With a cascading rail system, an overextension can be realized in particular, in which the carrier device or the sample platform is extended more than 100%, e.g. 101% to 120%, out of the chamber in a second position (P2). This makes it even easier to operate the carrier device or the sample platform.

The rail elements preferably comprise or consist of stainless steel. The pull-out mechanism, in particular the rail system, are in particular set up to remain in the chamber interior during the performance of a high-temperature process inside the chamber for sterilizing the chamber interior and also to be sterilized, wherein in this high-temperature process the chamber, the pull-out mechanism, in particular the rail system, are exposed to a temperature of between 150° C. and 200° C. for a period of several seconds to hours. Preferably, the pull-out mechanism is grease-free. Even if it is possible and preferred to use high-temperature-resistant greases to lubricate the bearings of the rail elements mounted on each other, the grease-free design is a particularly preferred embodiment.

Preferably, the pull-out mechanism comprises a pull-out base, also referred to as the base of the pull-out mechanism, in particular a base plate, also referred to as a sub-platform, which in particular supports the rail system by attaching it in particular to the pull-out base. The pull-out base is preferably mounted/fastened to at least one connecting element of a shaking device arranged in the chamber, in particular it can be detached without tools.

Preferably, the pull-out base, in particular the base plate, comprises at least one bearing section, preferably a plain bearing section, along which at least one complementary bearing section or plain bearing section of the carrier device or a sub-platform slides or rolls when the pull-out mechanism is pulled out. A plain bearing section can be a plastic part that is particularly abrasion-resistant and enables low-friction sliding. Temperature-resistant and/or sliding-optimized plastics are preferred. The plastic part can contain or consist of PEEK, polyoxymethylene or polyamide-66. The complementary sliding bearing section can be the outside of a base plate or a sub-platform of the pull-out mechanism.

The securing mechanism is used to lock and unlock the first position (P1). In the secured position (P1), the carrier device is prevented in particular from being pulled out in the pullout direction; in the unsecured position (P1), this is made possible in particular.

The securing mechanism can be unlocked by simultaneously actuating the actuating element and pulling the handle in the pull-out direction (A). Preferably, the securing mechanism can be unlocked by moving the handle from the first handle position (S1) to the second handle position (S2). This is achieved in particular by moving a connecting element, which is moved by the handle and in particular coupled to the carrier device and/or to the handle, from a first position (V1), in which the connecting element connects the carrier device to the pull-out base and/or to the chamber, to a second position (V2), in which the connecting element no longer connects the carrier device to the pull-out base and/or to the chamber.

The connecting element can be firmly and immovably connected to the handle or can be movably connected to the handle. The connecting element may be part of a connecting device, which may in particular have a connecting mechanism. More than one such connecting element may be provided, in particular two or more. The at least one connecting element can be an integral part of the handle, in particular the handle lever. The handle itself, in particular the handle lever, or the actuating element of the locking device or a part connected to it, can form the connecting element, in particular in that the pull-out base or the chamber comprises a stop element which, in the first handle position (S1), prevents the carrier device from being transferable from the first position (P1) to the second position (P2) by striking the connecting element against the stop element.

Preferably, the securing mechanism comprises a connecting device which comprises a movably arranged connecting element, in particular movably connected to the handle, for establishing and releasing a detachable connection of the carrier device and a base of the pull-out mechanism (“pull-out base”) in the first position (P1). In particular, the connecting element is arranged movably relative to the pull-out base and/or the carrier device and/or the handle. In particular, the connecting element can be arranged immovably on the handle.

Preferably, the securing mechanism comprises a connecting device which comprises a movably arranged connecting element, in particular movably connected to the handle, for establishing and releasing a releasable connection of the carrier device and a base of the pull-out mechanism in the first position (P1). Preferably, in particular by positioning (V1, V2) the connecting element in a first handle position (S1), the detachable connection of the carrier device and the base of the pull-out mechanism is established in a first position (V1) of the connecting element and released in a second handle position (S2) and second position (V2) of the connecting element.

Preferably, the handle (40) is arranged to move the connecting element (52), in particular by moving it (B) or by actuating the actuating element, so that in a first handle position (S1) the detachable connection of the carrier device (12) and the base (11) of the pull-out mechanism (10) is established and in a second handle position (S2) it is released.

Preferably, the handle is set up to move the connecting element by its movement, so that in a first handle position (S1) the detachable connection of the carrier device and the base of the pull-out mechanism is established and in a second handle position (S2) it is released.

Preferably, the locking device is set up so that the actuating element can be moved independently of the connecting element. In this case, which is described in the figures, actuation of the actuating element does not yet result in the connection between the carrier device and the pull-out base caused by the connecting element being released. In this way, a securing mechanism can be formed by means of the actuating element and a latch section connected thereto, which secures the handle position S1 to the carrier device, and in this way acts in addition to a securing mechanism formed by means of the connecting element to secure the first position P1, in that the carrier device is connected to the pullout base in the first position P1 by the connecting element and is only released by the movement, in particular pivoting movement, of the handle. This has the advantage that the force required to hold a total mass (pull-out, carrier device, sample platform and samples) does not have to be applied by a latch section, but is applied by the independent connecting element. Accordingly, a spring-induced restoring force of the actuating element can be configured to be lower, so that the actuation of the actuating element caused by one or a few fingers of the user is comfortable.

However, it is also possible and preferred that the locking device is set up so that the actuating element can be moved as a function of the connecting element. The actuating element, which preferably forms part of the handle or is arranged on the handle base or on the carrier device, can be set up to move the connecting element by its actuating movement, so that in a first handle position (S1) the detachable connection of the carrier device and the base of the pull-out mechanism is established and in a second handle position (S2) it is released. Preferably, the actuating element is coupled to the connecting element, in particular movably or immovably or integrally connected. Preferably, actuation of the actuating element leads to release of the detachable connection between the carrier element and the pull-out base. Preferably, the connecting element is released from the pullout base by actuating the actuating element. In this case, the actuating element preferably acts as a latch of the locking device, which in particular locks the carrier device to the pullout base, or possibly to the chamber or a component connected to the chamber, in particular a movably connected component. In particular, the component can enable the shaking movement while the first position 1 is secured.

The handle is in particular a component that is pivotably attached to the carrier device, with the pivot axis running in particular perpendicular to the pull-out direction.

The actuating element is in particular a spring-loaded component that can be operated by at least one finger of the user and is mounted in particular on the handle. The actuating element can be connected to the connecting element by at least one coupling element, in particular by a gear and/or a cable pull.

Preferably, the securing mechanism and/or the connecting device is set up so that when the handle is moved from the first handle position (S1) to the second handle position (S2) by manual operation of the handle, a tension existing in a dead center position must be overcome when the handle is moved between the first handle position (S1) and the second handle position (S2). The tension existing in the dead center position forms a maximum force that must be applied by the user by moving the handle. This design prevents the handle from releasing itself in the first position P1 during the shaking movement, even in the event that the locking device, which operates independently of the connecting element, is accidentally released. This advantage can also be achieved by a toggle lever arrangement instead of a mechanism implementing the spring element, in particular a dead center spring.

Preferably, the connecting device comprises at least one spring element, which is preferably arranged on the handle and can be tensioned in such a way that when the handle is moved from the first handle position (S1) to the second handle position (S2) by manual operation of the handle, a tension generated by the at least one spring element must be overcome when the handle is moved between the first handle position (S1) and the second handle position (S2).

Alternatively or additionally, the securing mechanism preferably includes a toggle lever arrangement, by means of which a dead center position of the handle is made possible. In particular, the toggle lever arrangement comprises a toggle lever. When the toggle lever is extended beyond the dead center position, a locking effect is achieved, in particular by providing a mechanical stop for the toggle lever on the carrier device. Even if the locking device is accidentally released, the locking effect of the toggle lever arrangement is maintained and the braced carrier device cannot release automatically.

Preferably, the at least one spring element is arranged between the connecting element and the handle in such a way that in the first handle position (S1), the connection of the carrier device and the base is secured by the tension of the at least one spring element.

Preferably, the at least one spring element is arranged between the connecting element and the handle in such a way that a dead center position of the handle is established by means of the spring element, which must be overcome in particular by manual operation of the handle when it is moved between the first handle position (S1) and the second handle position (S2), Preferably, the tension of the at least one spring element prevents the handle from pivoting into the second handle position (S2) by providing a dead center handle position between the first (S1) and second handle position (S2), in which the dead center spring in particular is under maximum tension.

Preferably, the locking device can be locked in the second handle position (S2), so that a displacement of the second handle position (S2) can be blocked by the locking device, and the locking device can be unlocked in the second handle position by manual actuation of the actuating element, so that the handle can be moved into the first handle position (S1).

Preferably, the handle is pivotable about a first pivot axis (X1), which is located in particular on the carrier device, and preferably the connecting element is pivotable about a second pivot axis (X2), which is located in particular on the handle and which is arranged on the handle truly parallel to the first pivot axis (X1). In this way, an efficient kinematic coupling of the handle movement with the movement of the connecting element required for securing/unsecuring can be achieved.

Preferably, a constrained guide section with a constrained guide is provided on the carrier device. Preferably, the connecting element comprises a guide element that can be guided by the constrained guide in the first position (P1) of the carrier device. Preferably, the guide element can be arranged in a first position (R1) relative to the constrained guide, in which the connection of the carrier element and a base of the pull-out mechanism is established, and in particular in a second position relative to the constrained guide, in which the connection of the carrier element and the base of the pull-out mechanism is released. Preferably, the guide element is guided from the first relative position to the second relative position by the constrained guide when the handle is moved manually from the first handle position (S1) to the second handle position (S2).

Preferably, the constrained guide is arranged such that the movement of the guide element in the constrained guide runs in a plane (E) to which a pivot axis (X1) of the handle runs perpendicular. Preferably, the guide element is arranged in the first relative position (R1) in an end section of the constrained guide extending along a y-direction and preferably the constrained guide is shaped to guide the guide element in a negative z-direction, when the handle (40) is moved from the first handle position (S1) into the second handle position (S2), in particular by the constrained guide extending obliquely downwards in the z-direction starting from the end section extending along the y-direction, whereby in particular the connecting element is released from an abutment element, in particular a hook element.

Preferably, an abutment element is fixedly connected to the base of the pull-out mechanism, in particular a hook element, on which the connecting element is supported in the first gripping position (S1) in order to form the connection between the carrier device and the base of the pull-out mechanism and to prevent the translational relative movement of the carrier device and the base.

Preferably, the guide element is supported on the abutment element in the first gripping position (S1), in particular by a positive connection suitable for transmitting force to form the connection of the carrier device to the base of the pull-out mechanism.

Preferably, the pull-out mechanism comprises a base. Preferably, at least one fastening part is provided to which the base is fastened. Preferably, the chamber comprises a chamber base with at least one opening, wherein in particular a fastening part is arranged in each case within an opening and is connected to the shaking device, so that an oscillating movement of the fastening part parallel to the chamber base is possible.

Preferably, the laboratory shaker comprises the sample platform, wherein the handle comprises at least one retaining section, in particular first bearing portion, and wherein the securing mechanism comprises at least one first stopper section, in particular abutment portion, which is fixedly connected to the base (11), wherein in particular the carrier device is secured in the first position (P1) of the carrier device and in the first handle position (S1) of the handle by the sample platform being positioned at the at least one positioning element and being held between the at least one retaining section of the handle and the at least one first stopper section of the base.

Preferably, the chamber comprises a chamber floor and preferably the pull-out mechanism comprises a base and at least one pull-out element, which in particular forms a component of a rail system of the pull-out mechanism and which is arranged movably on the base along the pull-out direction (A), and wherein the carrier device is fastened to at least one pull-out element. Preferably, the laboratory shaker comprises a fastening device which comprises at least one fastening part, in particular arranged on the chamber base and connected to the shaking device, and at least one connecting part, with which the base can be detachably fastened to the at least one fastening part, wherein the at least one fastening part and the at least one connecting part are set up for manual and tool-free fastening.

The invention also relates to a laboratory shaker for shaking samples stored in sample vessels, in the embodiment also shown here:
a temperature-adjustable chamber with a chamber opening,
a carrier device for carrying a sample platform,. the sample platform for carrying sample vessels, which is arranged on the carrier device,
a pull-out mechanism fixed in the chamber for the pull-out of the carrier device, which in a first position P1 is arranged completely in the chamber, which during manual pull-out passes through the chamber opening along a pull-out direction A and which in a second position P2 is arranged outside the chamber, wherein the pull-out mechanism comprises a base and at least one pull-out element which is arranged movably on the base along the pull-out direction A, and wherein the carrier device is fastened to the at least one pull-out element, which in particular forms a component of a rail system of the pull-out mechanism, and wherein the carrier device and/or the at least one pull-out element comprises at least one positioning element, by means of which the sample platform can be positioned on the carrier device,
a shaking device for moving the pull-out mechanism and the carrier device in the first position P1,
   wherein the laboratory shaker comprises a securing mechanism for securing the first position P1 of the carrier device and the securing mechanism includes a handle which is attached to the carrier device, which is movable between a first handle position S1 and a second handle position S2, wherein the carrier device and/or the handle comprises at least one retaining section, in particular a first bearing section, in particular a second stopper section, and wherein the securing mechanism comprises at least one first stopper section, in particular an abutment section, which is firmly connected to the base,
   wherein the carrier device is secured in the first position P1 of the carrier device and in the first handle position S1 of the handle by positioning the sample platform on the at least one positioning element and holding the carrier device and/or the sample platform between the at least one retaining section and the at least one first stopper section of the base. These embodiments of the laboratory shaker can be combined with all other optional embodiments of the laboratory shaker described herein.

The invention also relates to a laboratory shaker for shaking samples stored in sample vessels, in the embodiment also shown here:
a temperature-adjustable chamber with a chamber base and a chamber opening,
a carrier device for supporting a sample platform on which the sample vessels can be placed,
an extraction mechanism for the extraction of the carrier device, which in a first position P1 is arranged completely in the chamber, which during manual extraction passes through the chamber opening along an extraction direction A and which in a second position P2 is arranged outside the chamber,
a shaking device, in particular for moving the pull-out mechanism and the carrier device in the first position P1,
   wherein the pull-out mechanism comprises a base and at least one pull-out element, which in particular forms a component of a rail system of the pull-out mechanism, and which is arranged movably on the base along the pull-out direction A, and wherein the carrier device is fastened to the at least one pull-out element,
   wherein the laboratory shaker comprises a fastening device which comprises at least one fastening part, in particular arranged on the chamber base and connected to the shaking device, and at least one connecting part, with which the base can be detachably fastened to the at least one fastening part, wherein the at least one fastening part and the at least one connecting part are set up for manual and tool-free fastening. “Tool-free” means that, in addition to the connecting device, in particular apart from the two components “fastening part” and “connecting part”, no tool is required to release and/or establish the connection. In particular, the connecting device comprises an integrated tool, e.g. a lever or an operating wheel, which is non-detachably connected to the fastening part or the connecting part. These embodiments of the laboratory shaker can be combined with all other optional embodiments of the laboratory shaker described here. As soon as the fastening is tool-free, the fastening is also released without tools. This comprises the advantage for the user of being able to detach and remove the base from the chamber very quickly without the use of tools. The chamber is then particularly easy to clean. The parts can also be dismantled very quickly.

The laboratory shaker for shaking laboratory samples is configured in particular for tempering laboratory samples. Such devices are electrically operated and have a voltage connection. The laboratory shaker regulates the temperature of the laboratory samples, i.e. it keeps the inside of the housing and thus the laboratory samples stored there within tolerances by regulating the temperature to a target temperature that can be set by the user in particular. This can be above the room temperature (ambient temperature), as is the case with a heating cabinet or incubator, or below the room temperature, as is the case with a refrigerator or freezer. In a laboratory shaker configured as a climatic laboratory shaker, a climatic parameter prevailing inside the housing is preferably also controlled within tolerances. This climatic parameter can be the air humidity and/or a gas concentration, e.g. a CO₂, O₂ and/or N₂ concentration. Such a climatic laboratory shaker is, for example, a laboratory shaker for shaking laboratory samples, in particular with living cell cultures, with an incubator function, also known as an incubation shaker.

Typical features of such laboratory shakers can be one or more of the following: Temperature controllability of the chamber: cooling to at least 4° C., heating to a maximum of 80° C.
Speed range of the shaking movement: (25-500 rpm).
Housing format such that it can be set up in the laboratory (on the laboratory bench, under the laboratory bench, stackable stand models).
Stackability of the housing (2 or 3 or more on top of each other).
Capacity and throughput: container type, size and capacity.
Loading method (from the front or from above).
CO₂ regulation.
Photosynthetic light.

Particularly preferably, the laboratory shaker is set up to carry out a high-temperature process inside the chamber for sterilizing the chamber interior with the aid of a temperature control device and/or a heating device, in which the chamber is subjected to a temperature of between 150° C. for a period of several seconds (e.g. from 1, 2, 5, 10, 30 seconds) to several minutes (e.g. up to 1, 2, 3, 5, 10, 30, 60, 120, 240, 480 or 600 minutes) at a temperature of between 150° C. and 200° C., preferably at least 180° C., without the pull-out mechanism, preferably including the mounted sample platform, having to be removed. Particularly preferably, the laboratory shaker, in particular an electronic control device, which controls in particular the temperature control device and/or the heating device, is set up to expose the chamber to a target temperature of between 150° C. and 200° C., preferably at least 180° C., for a period of more than one hour, in particular for a period of several hours, e.g. for a period within a time interval of 2, 3, 4, 5, 6, 7, 8, 9 or 10 hours. The chamber generally requires a heating time to reach the target temperature and a cooling time to cool down from the target temperature to a normal operating temperature. In particular, the pull-out mechanism is located in the sealed chamber when the high-temperature cycle is carried out. The pull-out mechanism and the sample platform are made of a material that is resistant to high temperatures, in particular stainless steel.

The laboratory shaker preferably comprises a housing. The housing is preferably an outer housing whose housing walls are in contact with the environment. Accordingly, the housing door can be an outer housing door that is adjacent to the surroundings in the closed position.

In particular, the housing door comprises a hinge device that pivotably connects the housing door to the housing. Such a hinged door is moved by rotation between an open position and the closed position. In particular, the hinge device can be located on the vertically oriented outer edge of a cuboid housing-in the intended use of the laboratory shaker-which is adjacent to the housing opening. In the intended use of the laboratory shaker, the base plate of a cuboid housing is arranged horizontally, the side walls of the housing are arranged vertically in particular, and the top plate of the housing is arranged horizontally, in particular opposite the base plate.

A data processing device is preferably part of the electrical control device, which controls the functions of the laboratory shaker and which the laboratory shaker preferably has. The functions of the control device are implemented in particular by electronic circuits. The control device can have a microcontroller, a computing unit (CPU) for processing data and/or a microprocessor, each of which can contain the data processing device. The control device and/or the data processing device is preferably configured to implement a control method, which is also referred to as control software or control program. Such a control method can define the temporal course of a shaking movement which can be carried out by means of the shaking device. This shaking movement is defined in particular by direction(s) of translational movements and/or amplitude(s) of successive movement sections, which are carried out in an x-y plane. This x-y plane is usually parallel to the sample platform and/or a chamber bottom. Preferred diameters of a shaking movement carried out in an x-y plane are between 0 and 5 cm. The shaking device, in particular an orbital drive, is preferably set up for a shaking movement with a maximum diameter of between 0 and 5 cm. The functions of the laboratory shaker and/or the control device can be described in process steps. They can be implemented as components of the control program, in particular as subroutines of the control program.

Preferably, the laboratory shaker is an incubator shaker. The incubator shaker can then also be operated as a laboratory incubator and is therefore a device that can be used to create and maintain controlled climatic conditions for various biological development and growth processes. In particular, it is used to create and maintain a microclimate with controlled gas and/or humidity and/or temperature conditions in the chamber, whereby this treatment can be time-dependent. The incubation shaker can in particular have a timer, in particular a timer, a temperature control device configured as a heating and/or cooling device and preferably a setting for regulating an exchange gas supplied to the chamber, an adjustment device for the composition of the gas in the chamber of the incubation shaker, in particular for adjusting the CO₂ and/or the O₂ and/or the N₂ content of the gas and/or an adjustment device for adjusting the humidity in the chamber of the incubation shaker.

In particular, the incubator shaker comprises the incubator chamber (=chamber), and also preferably a control device with at least one control circuit, to which the at least one temperature control device is assigned as an actuator and at least one temperature sensor is assigned as a measuring element. Depending on the embodiment, the air humidity can also be controlled via this, although the air humidity itself is not measured by an air humidity sensor (rH sensor) and the air humidity is not an input variable of the control circuit. A tray filled with water in the incubator chamber can be heated or cooled in order to adjust the humidity via evaporation. CO₂-incubator shakers are used in particular for cultivating animal or human cells. Incubation shakers can have turning devices for turning the at least one cell culture container.

The control device can be set up so that a program parameter or a control parameter of the laboratory shaker, in particular the incubation shaker, is automatically selected as a function of other data. A treatment of the at least one cell culture in at least one cell culture container controlled by a control parameter corresponds in an incubator in particular to a climate treatment to which the at least one cell culture is subjected. Possible parameters, in particular program parameters, in particular user parameters, which are used to influence a climate treatment, define in particular the temperature of the chamber in which the at least one sample is incubated, the relative gas concentration of O₂—and/or CO₂ and/or N₂ in the chamber, the humidity in the chamber and/or at least one sequence parameter which influences or defines the sequence, in particular the order, of an incubation treatment program and/or shaking program consisting of several steps.

The temperature control device can be a combined heating/cooling device. It is preferably only a heating device. In particular, this can generate the heat via an electrical resistance wire. Preferably, the resistance wire is attached to the outside of at least one, several or all of the chamber walls forming the chamber.

The laboratory shaker or incubator shaker can have exactly one chamber, but can also have several chambers whose atmosphere (temperature, relative gas concentration, humidity) can be adjusted individually or collectively. A typical size of the interior of a chamber is between 50 and 400 liters, whereby smaller chamber sizes are also possible for special applications (IVF), in particular 10 to 49 liters.

Further preferred embodiments of a laboratory shaker according to the invention can be found in the description of the embodiments according to the figures.

It shows:

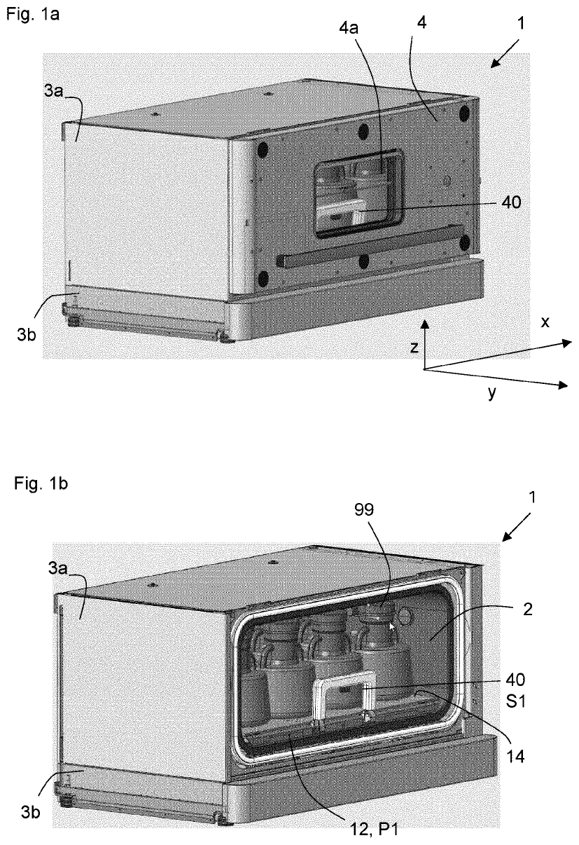

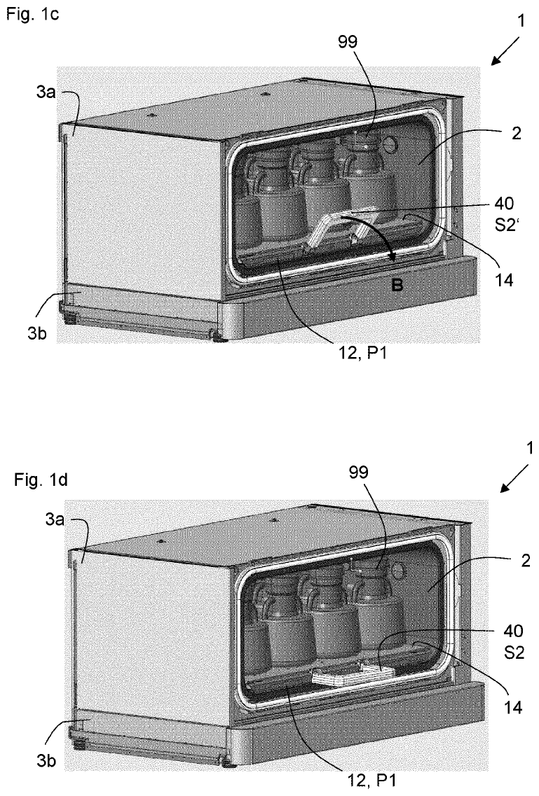

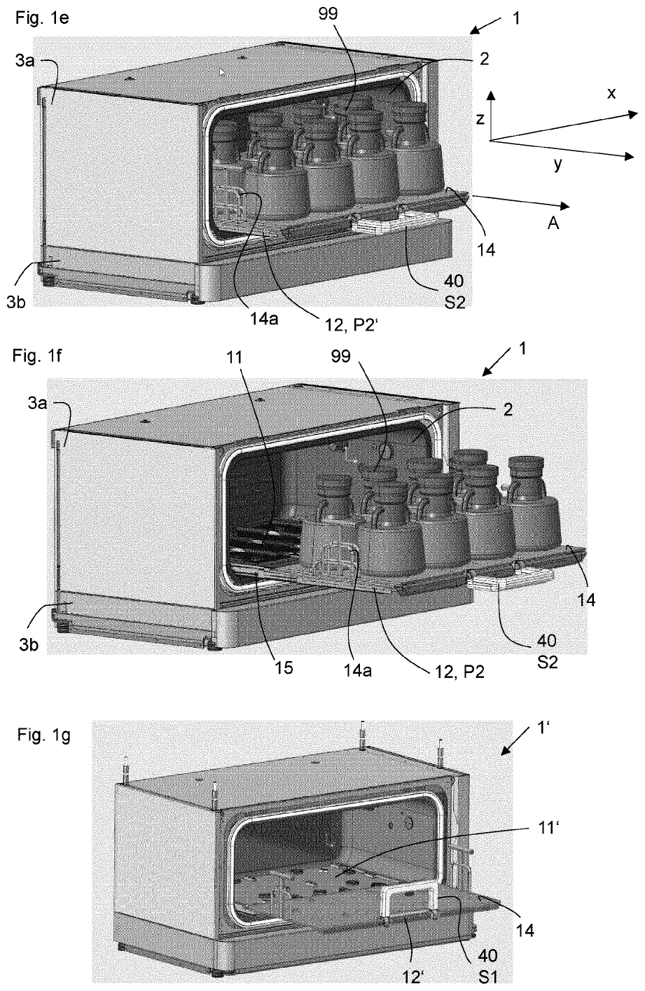

FIG. 1a shows a perspective side-frontal view of a laboratory shaker according to the invention according to an embodiment example, in the closed state of the housing door.

FIG. 1b shows the laboratory shaker of FIG. 1a, in the state with the housing door removed and filled with sample vessels, with handle position S1 of a handle of the carrier device of the pull-out mechanism arranged in position P1.

FIG. 1c shows the laboratory shaker from FIG. 1b in the state with handle position S2′ of the handle of the carrier device of the pull-out mechanism arranged in position P1.

FIG. 1d shows the laboratory shaker from FIG. 1b in the state with handle position S2 of the handle of the carrier device of the pull-out mechanism arranged in position P1.

FIG. 1e shows the laboratory shaker from FIG. 1d in the state with the carrier device of the pull-out mechanism partially pulled out and arranged in position P2′.

FIG. 1f shows the laboratory shaker from FIG. 1d in the state with the carrier device of the pull-out mechanism fully extended and arranged in position P2.

FIG. 1g shows a modified laboratory shaker similar to FIG. 1f, in the state with the handle in position S1 and with the carrier device of the pull-out mechanism fully extended and arranged in position P2.

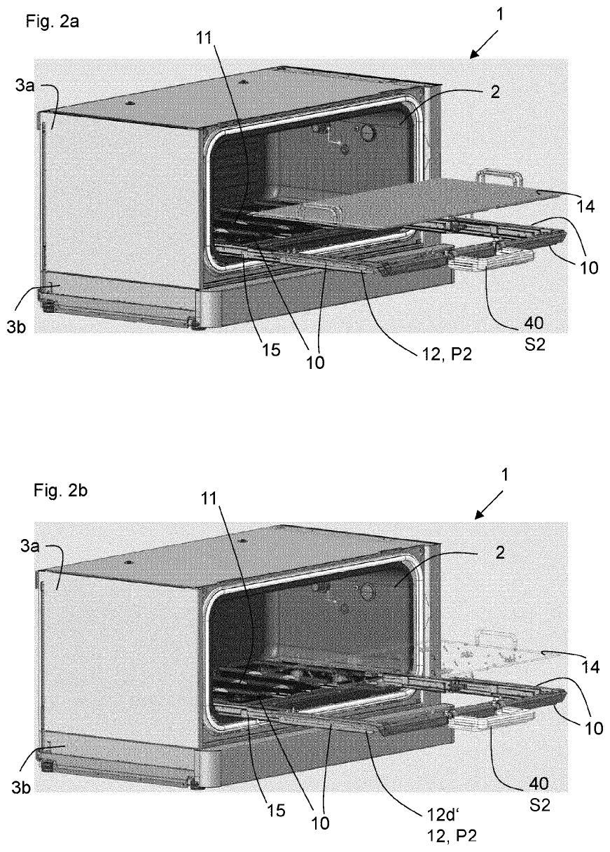

FIG. 2a shows the laboratory shaker from FIG. 1f, without sample vessels and with the sample platform removed upwards from the carrier device.

FIG. 2b shows the laboratory shaker from FIG. 2a, with the sample platform hidden.

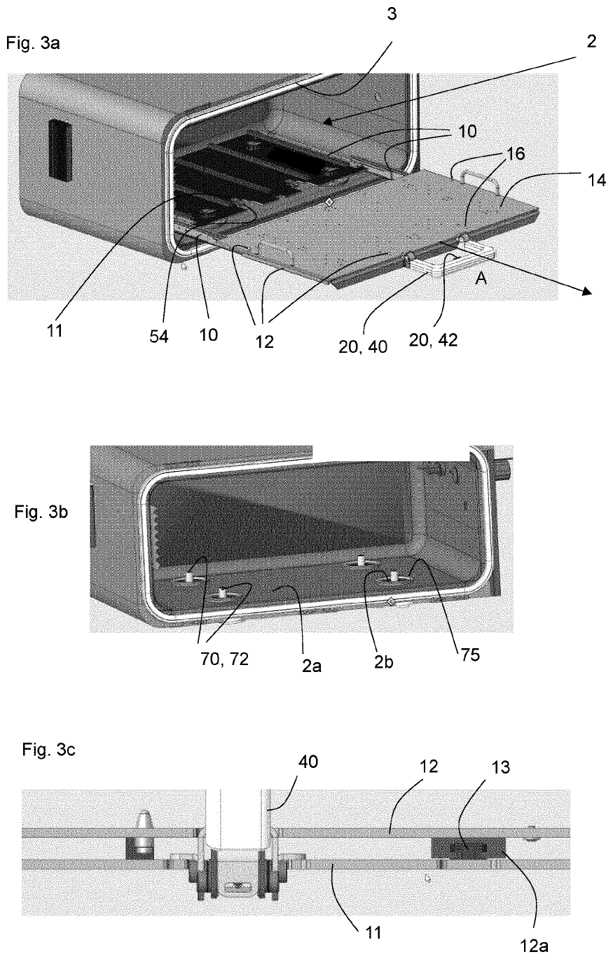

FIG. 3a corresponds to FIG. 1f, with the sample vessels removed from the sample platform.

FIG. 3b corresponds to FIG. 3a, with the pull-out mechanism including its base, rail system, carrier device and sample platform removed, corresponding to the state after tool-free disassembly for cleaning the chamber.

FIG. 3c shows a front view of the pull-out mechanism of the laboratory shaker of the previous figures, in which in particular the tongue and groove guide is visible, by means of which the translational constrained guide of the carrier device with respect to the base is secured.

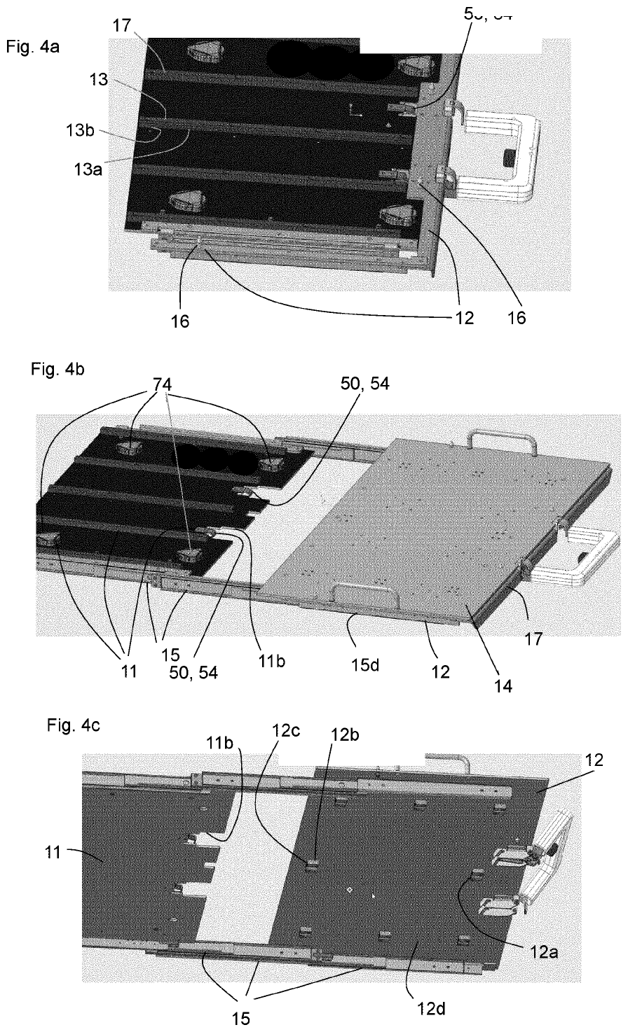

FIG. 4a shows a perspective side view of the pull-out mechanism of the laboratory shaker according to the previous figures, in the fully retracted state as in position P1.

FIG. 4b shows a side-frontal view of the pull-out mechanism of the laboratory shaker according to the previous figures, in the fully extended state as in position P2.

FIG. 4c shows a side-frontal bottom view of the pull-out mechanism of the laboratory shaker according to the previous figures, in the fully extended state as in position P2.

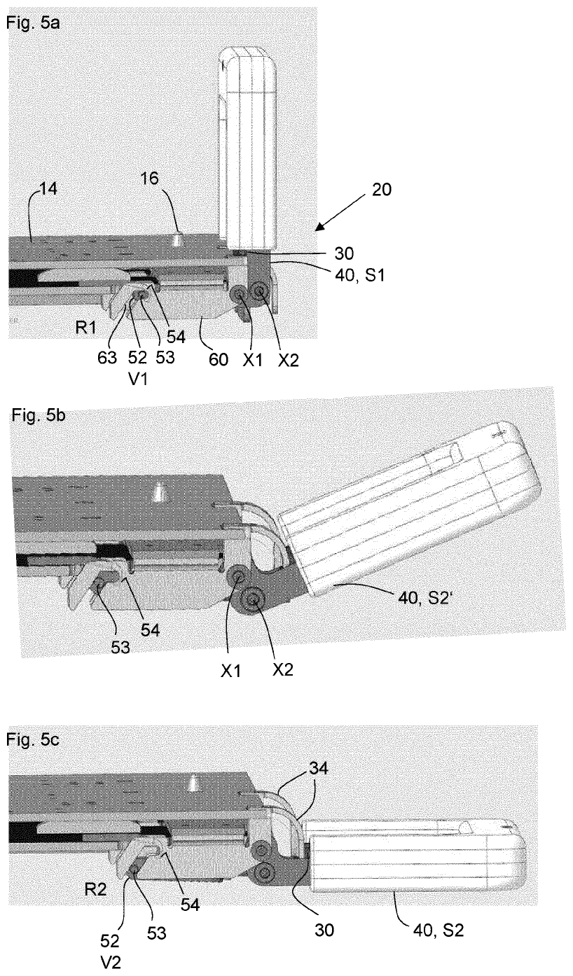

FIG. 5a shows a side perspective view of a detail of the carrier device of the laboratory shaker provided with a sample platform according to the previous figures, wherein the securing mechanism is shown, with which the first position P1 of the carrier device can be secured, with the handle of the securing mechanism in the vertical, first handle position S1, and with the relative position R1 of the guide element or the connecting element relative to the constrained guide.

FIG. 5b shows the view from FIG. 5a, with the handle of the securing mechanism in a handle position S2′, and with the relative position R2′ of the guide element or the connecting element relative to the constrained guide.

FIG. 5c shows the view from FIG. 5a, with the handle of the securing mechanism in a horizontal, second handle position S2, and with the relative position R2 of the guide element or the connecting element relative to the constrained guide.

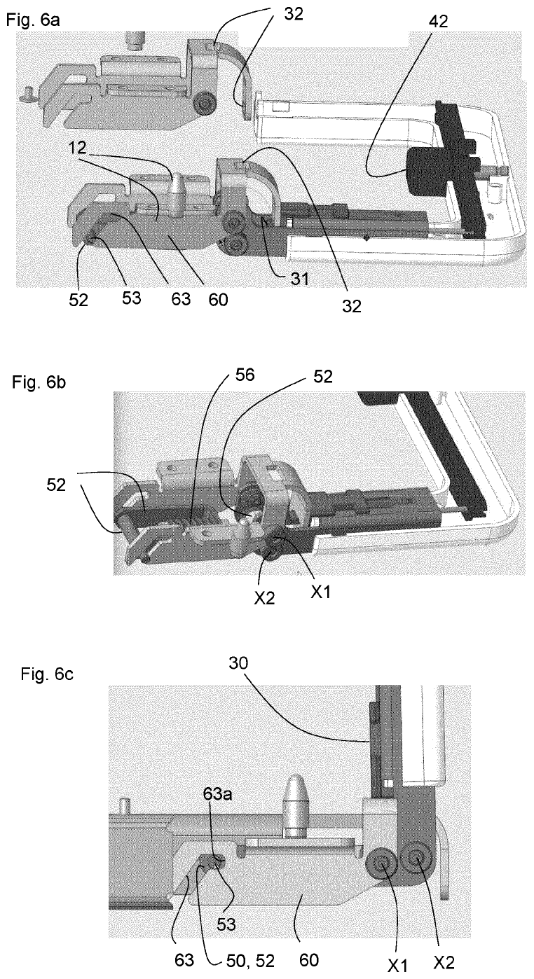

FIG. 6a shows the view from FIG. 5c, with further details.

FIG. 6b shows the view from FIG. 5c, with further details.

FIG. 6c shows the view from FIG. 5a, with further details.

FIG. 7 shows an optional detail of the laboratory shaker according to the previous figures, namely a prism-shaped first stopper section of the base of the pull-out mechanism, and the complementary prism-shaped second stopper section of the carrier device, wherein the carrier device strikes with the second stopper section against the first stopper section of the base in position P1 when the carrier device is moved from position P1 to position P1.

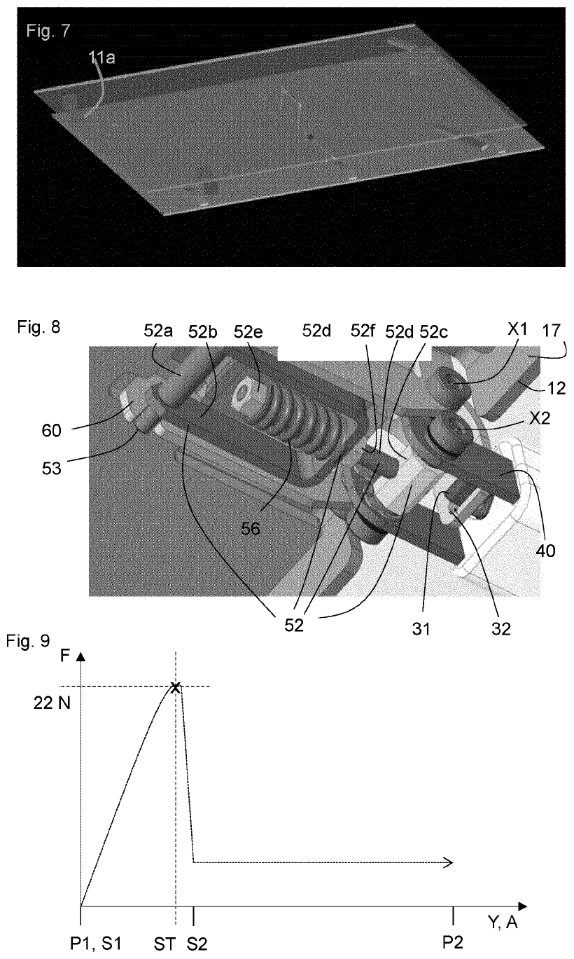

FIG. 8 shows a side perspective bottom view of a detail of the securing mechanism from FIGS. 5a to 5c.

FIG. 9 shows a displacement-force curve in which the force to be applied by the user is shown when the handle is moved from the first handle position to the second handle position and the carrier device is pulled by means of the handle in the pullout direction to a pull-out position, whereby this movement-apart from rotation-related components in the negative z-direction-takes place continuously in the pullout direction A.

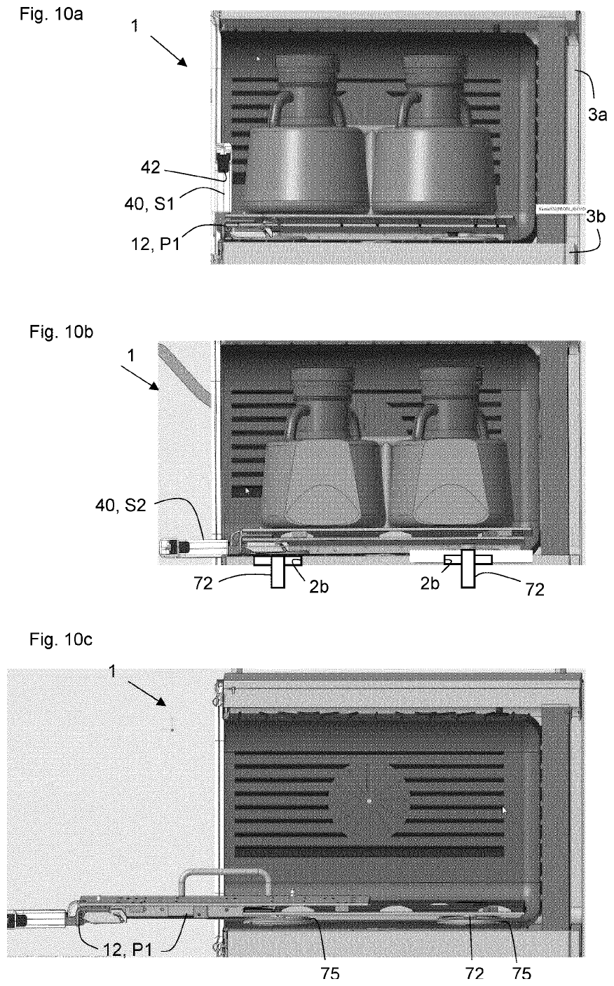

FIG. 10a shows a cross-sectional view along a y-z plane of the laboratory shaker in the state shown in FIG. 1b.

FIG. 10b shows a cross-sectional view along a y-z plane of the laboratory shaker in the state shown in FIG. 1d.

FIG. 10c shows a cross-sectional view along a y-z plane of the laboratory shaker of FIG. 1f in the state with partially extended carrier device, without sample vessels.

FIG. 11a shows a section of a carrier device and a handle section with connecting device of a securing mechanism according to a further embodiment of the laboratory shaker according to the invention, in connected position V1 of the connecting element, first position P1 of the pull-out and first handle position S1 of the handle.

FIG. 11b shows the handle section with the connecting device from FIG. 11a, in the released position V2 of the connecting element, first position P1 of the pull-out and second handle position S2 of the handle.

FIG. 11c shows a perspective view of the connecting device in FIG. 11b.

FIG. 11d shows the arrangement of the swivel axes of the connecting element, the locking lever and the swivel axis of the coupling point between the connecting element and the locking lever, as well as the swivel axis of the handle on the carrier device, in the relative position of these swivel axes in the dead center position shown in FIG. 11a.

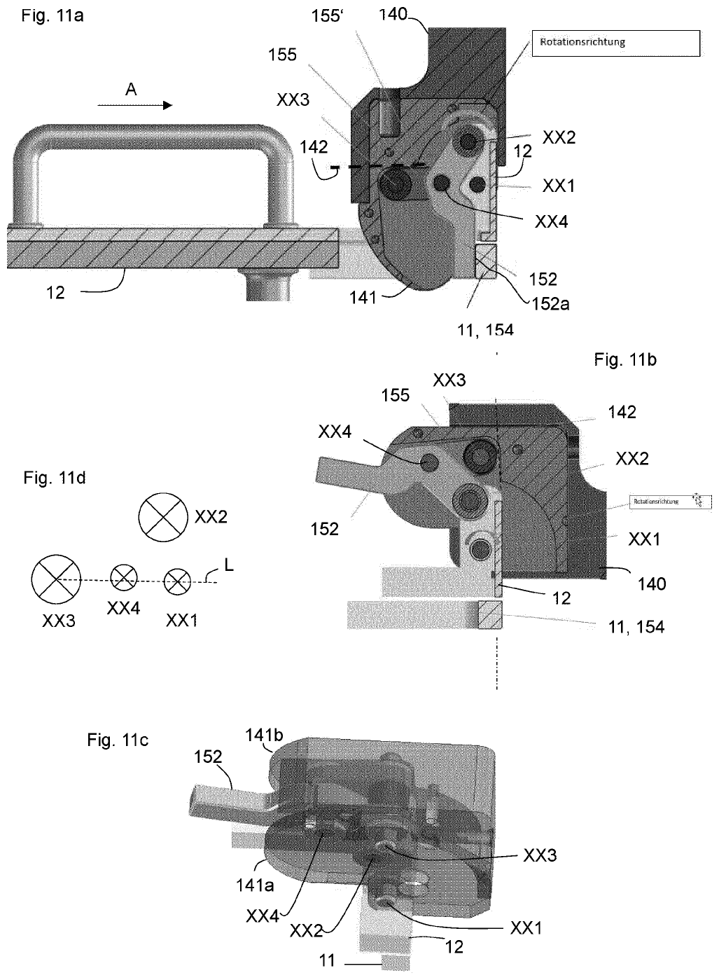

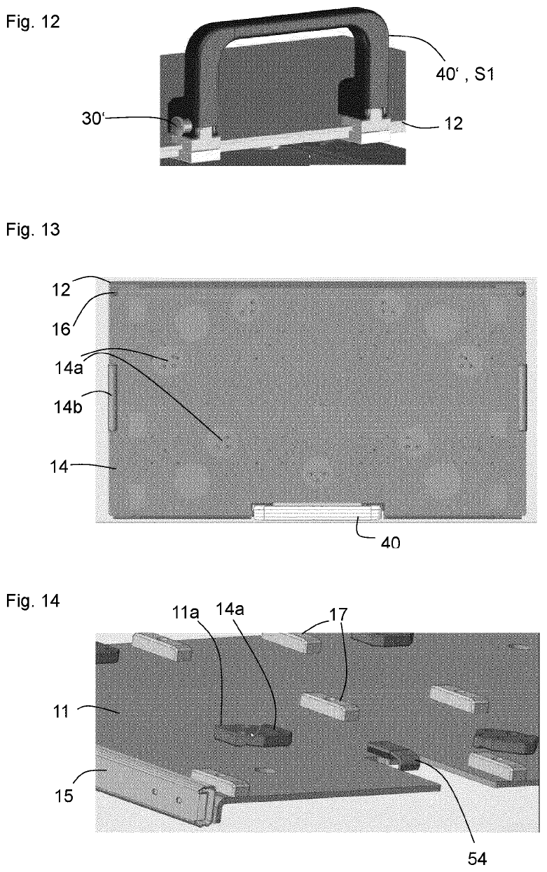

FIG. 12 shows a further embodiment example of the locking device of a securing mechanism of a laboratory shaker according to the invention, in a perspective lateral oblique detailed view.

FIG. 13 shows a sample platform with stop elements for attaching the sample platform and its carrier device to a pull-out base of a laboratory shaker according to the invention, in a perspective view from above.

FIG. 14 shows a base with stop elements for attaching the complementary stop elements of the sample platform of a laboratory shaker according to the invention shown in FIG. 13, in a perspective lateral oblique detailed view.

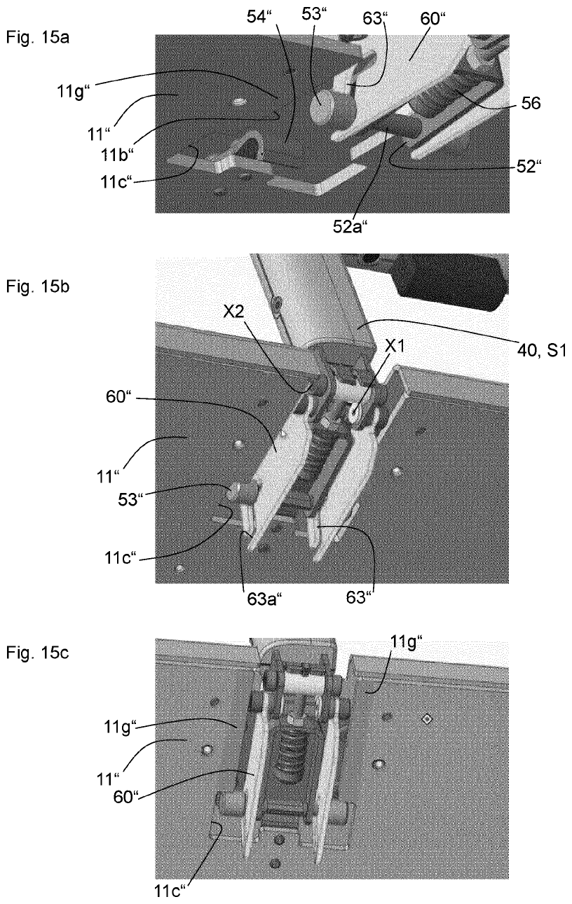

FIG. 15a shows a side perspective bottom view of a detail of the carrier device of the laboratory shaker provided with a sample platform according to a further preferred embodiment of the invention, wherein the securing mechanism is shown, with which the first position P1 of the carrier device can be secured, with the handle of the securing mechanism in the horizontal, second handle position S2, and with the relative position R2 of the guide element or the connecting element relative to the constrained guide.

FIG. 15b shows the view from FIG. 15a, with the handle of the securing mechanism in a vertical, first handle position S1, and with the relative position R1 of the guide element or the connecting element relative to the constrained guide.

FIG. 15c shows, similar to FIG. 15b, the view from FIG. 15a, with the handle of the securing mechanism in a vertical, first handle position S1, and with the relative position R1 of the guide element or the connecting element relative to the constrained guide.

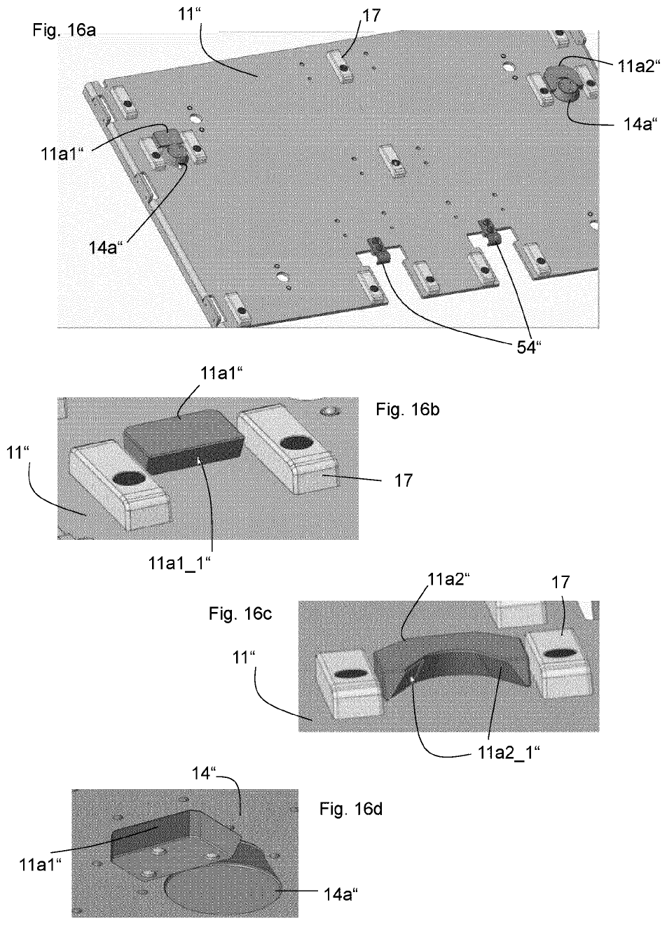

FIG. 16a shows a base with stop elements for attaching the complementary stop elements of a sample platform of a laboratory shaker according to the invention, in a perspective lateral oblique detailed view.

FIG. 16b and FIG. 16c each show a detailed section of the arrangement in FIG. 16a.

FIG. 16d shows a section of a carrier platform with the stop element mounted on it and the complementary stop element of the base.

In this example, the laboratory shaker 1 shown in the figures is an incubation shaker that can perform a shaking movement generated by an orbital drive, not explained in detail here, which is arranged below the chamber base 2a in the area of the housing section 3b (FIG. 1a). The samples to be shaken are located in laboratory vessels 99, which are placed on a sample platform 14. This can have, in particular removable, holders (not shown) for the sample vessels.

The sample platform is placed vertically from above on positioning pins 16 of the carrier device 12, so that the sample platform is essentially immovable, namely immovable within the x-y plane (in the intended use of the laboratory shaker the horizontal plane, i.e. perpendicular to the direction of gravity, FIG. 1a), and rests in the negative z-direction on the carrier device or on sections firmly connected to it.

The carrier device can be moved in translation along the pull-out direction A (y in the Cartesian coordinate system shown) by means of the pull-out mechanism 10, in particular its rail system, which comprises at least one, in this case several rails 15. The maximum amplitude of the translational mobility is defined by a stopper device, which is determined by sections of the base of the pull-out mechanism 10, in particular sections of the sub-platform 11 of the pull-out mechanism. A first stopper section 11a or 13b (FIG. 4a) is firmly connected to the sub-platform 11, i.e. the base 11. A second stopper section 12c is firmly connected to the carrier device, here in the form of the second stopper section 12c, which is attached to the underside of the carrier device or, in this case, to the underside of the sample platform 14 connected to the carrier device 12 (FIG. 4c; “downwards” refers to the direction of the negative z-axis; “forwards” refers to the direction of the positive y-axis).

The pull-out mechanism is set up here so that the carrier device 12 is essentially immovable in relation to the base 11 along the z-axis and along the x-axis, and only translatable. Movable along the y-axis. However, it is also possible that the pull-out mechanism is set up in such a way that the pull-out does not exclusively have a translational guide in only one direction, but instead, for example, performs a mixed translational pull-out movement in different translational directions, or a rotating pull-out movement, or a mixed translational-rotational pull-out movement, e.g. along the x-y plane.

The base 11 of the pull-out mechanism 10, and thus the sample platform mounted thereon, is connected to a shaking device, in particular an orbital drive (not visible), via a fastening device 70, which in particular includes fastening elements 72 (FIG. 3b) or nut elements 74 (FIG. 4b) that can be operated without tools and in particular have a fine thread. For this purpose, the fastening elements are each arranged within an opening 2b of the chamber base 2a within a diameter of e.g. 5 cm of the opening 2b and extend vertically from the area below the chamber base 2a into the area above the chamber base. There, the fastening elements 72 have flange sections for supporting the sub-platform 11 of the base of the pull-out mechanism. This arrangement enables shaking movements with deflections within the diameter range of up to 5 cm, for example. The entire pull-out mechanism is moved. This is facilitated by the fact that the design of the pull-out mechanism is sufficiently low-mass; in particular, the pull-out mechanism or the rail elements 15 are made of or consist of stainless steel.

FIG. 1a shows a perspective side-frontal view of a laboratory shaker 1 according to the invention according to an embodiment example, in the closed state of the housing door 4, which comprises a viewing window 4a through which the handle 40 of the carrier device 12 and the interior of the chamber 2 are visible. The housing 3a, 3b of the shaker 1 is essentially rectangular, as is the chamber interior.

The laboratory shaker 1 is configured for shaking samples stored in sample vessels 99. It has:
a temperature-adjustable chamber 2 with a closable chamber opening 3,
a carrier device 12 for supporting a sample platform 14, on which the sample vessels 99 are placed in FIG. 1a,
a pull-out mechanism 10 for the pull-out of the carrier device 12, which in a first position P1 (FIG. 1a, 1b) is arranged completely in the chamber 2, which during manual pull-out passes through the chamber opening 3 along a pull-out direction A and which in a second position P2 is arranged outside the chamber 2, in particular completely outside the chamber 2a (FIG. 1f), if the pull-out is, as preferred here, a full pull-out;
a shaking device for shaking the samples 99,
a securing mechanism 20 for securing and releasing the first position P1, with a locking device 30 and a handle 40, which is fastened to the carrier device 12 and is movable between a first handle position S1, in which the locking device 30 is locked, and a second handle position S2, in which the locking device 30 is unlocked; the handle is configured in particular as a pivoting lever, which is fastened here to the carrier device 12 so as to be pivotable about an axis X1. The carrier device 12 preferably has, in particular, rail elements 15, on which L-shaped strips 15d (FIG. 4b) are mounted, to which the positioning elements 16 are fastened here, and/or preferably comprises a front profile 17 (FIG. 4b), on which the handle 40 and a constrained guide section 60 are mounted.

A displacement of the first handle position S1 (FIG. 1b, 5a) can be blocked by the locking device 30. The locking device 30 comprises a manually operable actuating element 42 arranged on the handle 40, by actuating which in the direction towards the handle, i.e. in particular by one-handed operation, the locking device 30 can be unlocked in the first handle position S1, so that the handle 40 can be moved into the second handle position S2 (FIG. 1d, 5c), whereby the securing mechanism 20 can be unlocked by simultaneously actuating the actuating element 42 and pulling the handle 40 in the pull-out direction A. This results in a flowing movement sequence that is perceived as very ergonomic by users.

To unlock the securing mechanism 20, the spring-loaded button 42 simply needs to be pressed with the fingers of one hand, which unlocks the locking element 31 and the locking opening 32 on the carrier element via a pull mechanism. At the same time, the user pulls the handle in the pull-out direction so that the handle moves downwards; the button 42 can be released again immediately, which is convenient and intuitive, the handle automatically engages in the second handle position S2 due to the spring tension (FIG. 5c), and the user pulls out the carrier device 12 (including sample platform and vessels) with the same pullout movement with which he also locked the handle.

The securing mechanism 20 preferably comprises a connecting device 50 which comprises a movably arranged connecting element 52, in particular movably connected to the handle 40, for establishing and releasing a detachable connection of the carrier device 12 and a base 11 of the pull-out mechanism 10 in the first position P1. The connecting element is multi-part and is described in detail in FIG. 8. The handle 40 is preferably arranged to move the connecting element 52 by its pivoting movement B, so that in a first handle position S1 the releasable connection of the carrier device 12 and the base 11 of the pullout mechanism 10 is established and in a second handle position S2 it is released. In this way, the carrier device 12 is secured to the base. As an alternative or additional embodiment, it is preferred that the latch 31 of the locking device 30 of the handle 40 latches in position P1 of the pull-out mechanism or the carrier device and in particular in handle position S2 with a section 32′, which is firmly connected to the chamber or the chamber base. This embodiment is not shown here.

Preferably, the connecting device comprises at least one spring element 56, which is preferably arranged on the handle and can be tensioned in such a way that when the handle is moved from the first handle position S1 to the second handle position S2 by manual operation of the handle, a tension generated by the at least one spring element must be overcome when the handle is moved between the first handle position S1 and the second handle position S2. This secures the first handle position S1 and, due to the connection of the connecting element 52 to the base 11 (hook 54), also secures the basic position P1 of the pull-out mechanism. This embodiment is shown here.

Preferably, the at least one spring element 56 is arranged between the connecting element 12 and the handle 40 such that, in the first handle position S1, the connection of the carrier device 12 and the base 11 is secured by the tension of the at least one spring element 56. This embodiment is shown here.

Preferably, the at least one spring element 56 is arranged between the connecting element 52 and the handle 40 in such a way that a dead center position of the handle is established by means of the spring element 56 (as shown here), which must be overcome in particular by manual operation of the handle when it is moved between the first handle position (S1) and the second handle position (S2). This embodiment is shown here.

Preferably, the handle 40 can be secured against pivoting into the second handle position S2 by the tension of the at least one spring element 56, as implemented here by providing a dead center handle position lying between the first S1 and second handle position S2, in which the dead center spring 56 in particular is under maximum tension. This embodiment is shown here.

The term dead center is used when the connecting joints and the acting force vectors of a flat lever mechanism lie on a common straight line. The dead center position is present here in particular when the axes X1, X2 and the axis of the guide element 53, viewed in the y-z plane, lie on a straight line. The corresponding handle position is referred to as the dead center handle position ST.

The force applied is only transferred to the mechanism's holding point; the levers cannot be moved without external influence. Only a force transverse to the main axis of this mechanism changes this state. The dead center, which can only be reached and overcome against a spring force 56 (e.g. 22 N, see FIG. 8), is used to secure the home position P1; this is a self-securing mechanism. This design is shown here.

Preferably, the locking device 30 is lockable in the second handle position S2, so that a displacement of the second handle position S2 can be blocked by the locking device 30. Preferably, the locking device 30 can be unlocked in the second handle position S2 by manual actuation of the actuating element 42, so that the handle 40 can be moved into the first handle position S1. This embodiment is shown here.

Preferably, the handle (40) is pivotable about a first pivot axis (X1), which is located in particular on the carrier device. This embodiment is shown here. Preferably, the connecting element 52 is pivotable about a second pivot axis X2, which is located in particular on the handle 40, and which is arranged substantially parallel, i.e. at a distance, to the first pivot axis X1 on the handle 40. This embodiment is shown here.

Preferably, a constrained guide section 60 with a constrained guide 63, here in particular a link 63, is provided on the carrier device 12 and the connecting element 52, here in particular a tie rod with spring, preferably comprises at least one guide element 53, here in particular a link guide 53, which can be guided by the constrained guide 63 in the first position P1 of the carrier device 12, wherein the guide element 53 can be arranged in a first position R1 relative to the constrained guide 63, in which the connection of the carrier element 12 and a base 11 of the pull-out mechanism 10 is established, and can be arranged in a second position relative to the constrained guide, in which the connection of the carrier element 12 and the base 11 of the pull-out mechanism 10 is released, wherein the guide element 53 is guided by the constrained guide from the first relative position into the second relative position when the handle is moved manually from the first handle position S1 into the second handle position (S2). In this way, the securing mechanism 20 can be unlocked conveniently, intuitively and reliably. These embodiments are shown here.

Preferably, the constrained guide 63 is arranged such that the movement of the guide element 53 along the constrained guide 63 extends in a plane E to which a pivot axis X1 at a base of the handle 40 extends perpendicularly, and wherein the guide element 53 in the first relative position R1 is arranged in an end portion 63a of the constrained guide 63 extending along a y-direction, and wherein the constrained guide 63 is preferably shaped for this purpose, guiding the guide element 53 in a negative z-direction when the handle 40 is moved from the first handle position S1 into the second handle position S2, in particular by the constrained guide extending obliquely downwards in the z-direction starting from the end section extending along the y-direction, whereby in particular the connecting element (52) is released from an abutment element 54, in particular hook element 54. These embodiments are shown here.

Preferably, at least one abutment element 54 is firmly connected to the base 11 of the pullout mechanism, in particular a hook element 54, on which the connecting element 52 is supported in the first gripping position S1, in order to form the connection of the carrier device to the base 11 of the pull-out mechanism 10 and to prevent the translational relative movement of the carrier device 12 and the base 11. These embodiments are shown here.

Preferably, the guide element 53 is supported in the first gripping position S1 on the abutment element 54, in particular by a positive connection which is suitable for force transmission, in particular to form the connection of the carrier device 12 to the base 11 of the pull-out mechanism 10. These embodiments are shown here.

Preferably, the pull-out mechanism 10 comprises a base 11, which particularly preferably comprises a sub-platform 11 arranged parallel to the chamber base 2a and in particular to the sample platform 14, which can be used for mounting the pull-out mechanism 10 on the shaking device. Preferably, at least one fastening part 72 is provided, to which the base 11 is fastened. Preferably, the chamber 2 comprises a chamber base 2a with at least one opening 2b (here: 4 openings 2b). Preferably, a fastening part 70 is arranged within an opening 2b and, in particular, connected to the shaking device, so that an oscillating movement of the fastening part 72 parallel to the chamber base 2a and, in particular, of the associated assembly of pull-out mechanism 10 including carrier device 12 and sample platform 14 is possible. These embodiments are shown here.

Preferably, an insert element 75 is arranged in each of the openings 2b, which is hollow-cylindrical in shape at least in sections, projects beyond the chamber floor and surrounds the respective opening within which the fastening part 72 moves, preferably without contacting the hollow-cylindrical section of the insert element 75. The opening permits the shaking movement of the pull-out mechanism and all associated components, which is mediated by the fastening part 72.

Preferably, the handle comprises at least one retaining section 43, in particular a first bearing section 43, wherein the securing mechanism 20 preferably comprises at least one first stopper section 11a (complementary prism block) or 13b (stop element), in particular an abutment section, which is firmly connected to the base 11. In particular, the carrier device 12 is secured in the first position P1 of the carrier device 12 and in the first handle position S1 of the handle 40 by positioning the sample platform 14 on the at least one positioning element 16 and holding it between the at least one retaining section 43 of the handle 40 and the at least one first stopper section 11a; 13b of the base 11, in particular by applying force, in particular spring force 56 or another spring. In this way, the sample platform 14 in position P1 can be secured even better against displacement in the z-direction and y-direction. These embodiments are shown here for the most part.

Preferably, the chamber 2 comprises a chamber base 2a, wherein preferably the pull-out mechanism 10 comprises a base 11 and at least one pull-out element 15, which in particular forms a component of a rail system of the pull-out mechanism, and which is arranged movably along the pull-out direction A on the base 11, and wherein the carrier device 12 is fastened to at least one pull-out element 15. The laboratory shaker 1 preferably comprises a fastening device 70, which comprises at least one fastening part 72, in particular arranged on the chamber base 2a and connected to the shaking device, and at least one connecting part 74, with which the base 11 can be detachably fastened to the at least one fastening part 72, wherein the at least one fastening part 72 and the at least one connecting part 74 are set up for manual and tool-free fastening. These embodiments are shown here. This manual and tool-free fastening results in a simple and convenient way of cleaning the chamber, which makes working with the laboratory shaker efficient and safer. These embodiments are shown here. In particular, the connecting part 74 is configured to be screwed onto the fastening part, in particular by means of a fine thread. Alternatively, and preferably in each case, the fastening part 72 and/or the connecting part 74 can also be used for tool-free connectability and detachability: Single thread, multiple thread, bayonet; quick-release fastener; quick-release nut. This allows the connection to be realized conveniently, simply, securely and with durable means.

The invention also relates to a laboratory shaker 1 for shaking samples stored in sample vessels, in the embodiment also shown here:
a temperature-adjustable chamber 2 with a chamber opening 3,
a carrier device 12 for carrying a sample platform 14,
the sample platform 14 for carrying sample vessels, which is arranged on the carrier device 12,
a pull-out mechanism 10 fixed in the chamber 2 for the pull-out of the carrier device 12, which in a first position P1 is arranged completely in the chamber 2, which during manual pull-out passes through the chamber opening 3 along a pull-out direction A and which in a second position P2 is arranged outside the chamber 2, wherein the pull-out mechanism 10 comprises a base 11 and at least one pull-out element 15 which is arranged movably on the base 11 along the pull-out direction A, and wherein the carrier device 12 is fastened to the at least one pull-out element 15, which in particular forms a component of a rail system of the pull-out mechanism, and which comprises at least one positioning element 16, by means of which the sample platform 14 can be positioned on the carrier device 12,
a shaking device for moving the pull-out mechanism and the carrier device (12) in the first position (P1),
   wherein the laboratory shaker 1 comprises a securing mechanism 20 for securing the first position P1 of the carrier device 12 and the securing mechanism 20 includes a handle 40 which is fastened to the carrier device 12, which is movable between a first handle position S1 and a second handle position S2 and which comprises at least one retaining section 43, in particular a first bearing section 43, and wherein the securing mechanism 20 comprises at least one first stopper section 11a; 13b, in particular an abutment portion 11a; 13b, which is firmly connected to the base 11,
   wherein the carrier device 12 is secured in the first position P1 of the carrier device 12 and in the first handle position S1 of the handle 40 by positioning the sample platform 14 on the at least one positioning element 16 and holding it between the at least one retaining section 43 of the handle 40 and the at least one first stopper section 11a; 13b of the base 11. This embodiment of the laboratory shaker can be combined with all other optional embodiments of the laboratory shaker 1 described herein. In particular with the following embodiments:

Preferably, the at least one retaining section 43 of the handle 40 is pressed against the sample platform 14 in the first handle position S1 by a spring element 56 of the securing mechanism 20, in particular by a dead center spring 56, and this is pressed against the at least one second stopper section 11a; 13b serving as an abutment section via at least one second stopper section 12c firmly connected to the carrier device.

Preferably, the at least one second stopper section 11a is configured to form a tongue-and-groove connection (12c; 13) movable along the pull-out direction A, and/or comprises a ramp-shaped course along the pull-out direction A, with which the sample platform 14 can be fixed in the first position P1 with respect to a z-direction of the base 11.

The invention also relates to a laboratory shaker 1 for shaking samples stored in sample vessels, in the embodiment also shown here:
a temperature-adjustable chamber 2 with a chamber base 2a and a chamber opening 3,
a carrier device 12 for supporting a sample platform 14 on which the sample vessels can be placed,
a pull-out mechanism 10 for pulling out the carrier device 12, which is arranged completely in the chamber 2 in a first position P1, which passes through the chamber opening 3 along a pull-out direction A during manual pull-out and which is arranged outside the chamber 2 in a second position P2,
a shaking device, in particular for moving the pull-out mechanism and the carrier device 12 in the first position P1,
   wherein the pull-out mechanism 10 comprises a base 11 and at least one pull-out element 15, which in particular forms a component of a rail system of the pull-out mechanism, and which is arranged movably along the pull-out direction A on the base 11, and wherein the carrier device 12 is fastened to the at least one pull-out element 15,
   wherein the laboratory shaker comprises a fastening device 70 which comprises at least one fastening part 72, in particular arranged on the chamber base 2a and connected to the shaking device, and at least one connecting part 74, with which the base 11 can be detachably fastened to the at least one fastening part 72, wherein the at least one fastening part 72 and the at least one connecting part 74 are set up for manual and tool-free fastening. “Tool-free” means that, in addition to the connecting device, in particular apart from the two components “fastening part” and “connecting part”, no tool is required to release and/or establish the connection. In particular, the connecting device comprises an integrated tool, e.g. a lever or an operating wheel, which is non-detachably connected to the fastening part or the connecting part. These embodiments of the laboratory shaker can be combined with all other optional embodiments of the laboratory shaker 1 described here. In particular with the following embodiments:

Preferably, a fastening part 72 and a connecting part 74 can each be connected in a force-fit and/or form-fit manner, in particular by screwing, and preferably have a fine thread, thread, bayonet or quick release. Preferably, the fastening device 70 comprises at least one quick-action clamping device for fastening the base 11.

Preferably, the chamber comprises a chamber base 2a with at least one opening 2b, within which the at least one fastening part 72 is arranged movably parallel to the chamber base 2a, in particular for performing an orbital movement. This preferably includes translational movements; in particular, translations are superimposed in such a way that a circular movement is mapped on the entire base at each integral point; execution in particular according to an orbital mixer; in this case, an eccentric specifies an orbit and specifies the amplitude; in particular, a z-beat is avoided, it is preferably a planar translational movement in the x-y plane.

FIG. 1b shows the laboratory shaker 1 of FIG. 1a, in the state with the housing door 4 removed and filled with sample vessels 99, with handle position S1 of a handle of the carrier device of the pull-out mechanism 10 arranged in position P1. FIG. 1c shows the laboratory shaker 1 of FIG. 1b, in the state with handle position S2′ of the handle of the carrier device 12 of the pull-out mechanism 10 arranged in position P1. FIG. 1d shows the laboratory shaker 1 of FIG. 1b, in the state with handle position S2 of the handle of the carrier device 12 of the pull-out mechanism 10 arranged in position P1. FIG. 1e shows the laboratory shaker 1 of FIG. 1d, in the state with the carrier device 12 of the pull-out mechanism 10 partially pulled out and arranged in position P2′. FIG. 1f shows the laboratory shaker 1 of FIG. 1d, in the state with the carrier device 12 of the pull-out mechanism 10 fully pulled out and arranged in position P2.

FIG. 2a shows the laboratory shaker 1 of FIG. 1f, without sample vessels 99 and with the sample platform 12 removed upwards (in the positive z-direction) from the carrier device 12. FIG. 2b shows the laboratory shaker 1 of FIG. 2a, with the sample platform hidden.

FIG. 3a corresponds to FIG. 1f, whereby the sample vessels 99 have been removed from the sample platform 14. FIG. 3b corresponds to FIG. 3a, with the pull-out mechanism 10 including its base 11, rail system 15, carrier device 12 and sample platform 14 having been removed, corresponding to the state after tool-free disassembly for cleaning the chamber. FIG. 3c shows a front view of the pull-out mechanism 10 of the laboratory shaker 1 of the previous figures, in which in particular the tongue and groove guide (12c; 13) is visible, by means of which the translational constrained guide of the carrier device 12 with respect to the base 11 is secured.

FIG. 4a shows a lateral perspective view of the pull-out mechanism 10 of the laboratory shaker 1 according to the previous figures, in the fully retracted state as in position P1. FIG. 4b shows a side-frontal view of the pull-out mechanism 10 of the laboratory shaker 1 as shown in the previous figures, in the fully extended state as in position P2. FIG. 4c shows a side-frontal bottom view of the pull-out mechanism 10 of the laboratory shaker 1 according to the previous figures, in the fully extended state as in position P2.

FIG. 5a shows a side perspective view of a detail of the carrier device 12 of the laboratory shaker 1 provided with sample platform 14 according to the previous figures, wherein the securing mechanism 20 is shown, with which the first position P1 of the carrier device 12 can be secured, with the handle 40 of the securing mechanism 20 in the vertical, first handle position S1, and with the relative position R1 of the guide element 53 or of the connecting element 52 relative to the constrained guide 63. FIG. 5b shows the view from FIG. 5a, with the handle of the securing mechanism 20 in a handle position S2′, and with the relative position R2′ of the guide element 53 or of the connecting element 51 relative to the constrained guide 63. FIG. 5c shows the view from FIG. 5a, with the handle of the securing mechanism in a horizontal, second handle position S2, and with the relative position R2 of the guide element 53 or of the connecting element 52 relative to the constrained guide 63.

FIG. 6a shows the view from FIG. 5c, with further details. FIG. 6b shows the view from FIG. 5c, with further details. FIG. 6c shows the view from FIG. 5a, with further details.

FIG. 7 shows an optional detail of the laboratory shaker 1 according to the previous figures, namely a prism-shaped first stopper section 11a of the base 11 of the pull-out mechanism 10, and the complementary prism-shaped second stopper section of the carrier device, wherein the carrier device 12 abuts with the second stopper section against the first stopper section 11a of the base 11 in position P1 when the carrier device is moved from position P1 to position P1.

FIG. 8 shows a side perspective bottom view of a detail of the securing mechanism from FIGS. 5a to 5c. Shown in detail is the connecting element 52, which is movably connected here to the handle 40 via a pivot axis X2. It is used to establish and release a detachable connection between the carrier device 12 and a base 11 of the pull-out mechanism 10 in the first position P1 shown here. The connecting element comprises an axle member 52c, which is arranged in the shape of a bushing around the swivel axis X2. Perpendicular to the pivot axis X2, a pin element 52d is firmly connected to the axle member 52c. On this pin element, the spring element 56 is used for the spring-loaded mounting of a coupling element 52b, which is configured here as a clamp. The clamp 52b holds a coupling rod 52a at its end facing away from the axis X2. This coupling rod 52a runs parallel to the axis X2 and serves to engage in the hook 54 in order to enable the connection or securing of the carrier device 12 or the carrier platform 14 to the base 11. The spring-loaded mounting of the coupling element 52b on the pin element 52d is made possible here by external threads of the pin element and two nuts 52e and 52f, between which a spring 56-here wound around the pin element-and a section of the coupling element 52b are positioned. When the lever is raised, the spring 56 is compressed as the pin element 52d is pulled away from the coupling element 52e. In the extension of the coupling rod 52a, guide pins 53 are arranged on both sides, which engage in the constrained guide 60 when the handle is pivoted, whereby the coupling rod 52a is lifted up to the height of the hook.

FIG. 9 shows a displacement-force curve in which the force F to be applied by the user is shown when the handle is moved from the first handle position S1 to the second handle position S2 while passing the dead center handle position ST (measured at Fₘₐₓ=22N) and the carrier device 12 is pulled by means of the handle 40 in pull-out direction A to a pull-out position P2, this movement taking place continuously in pull-out direction A, apart from rotation-related components in the negative z-direction. Preferably, the securing mechanism and in particular the spring element 56 is set up such that the maximum pullout force Fₘₐₓ to be applied by the user when pulling the handle from the position S1 to the position S2 or in particular from the position S1 to the position P2 is preferably less than or equal to 50N, preferably 40 N, preferably 30 N, preferably 20 N, preferably 10N.

FIG. 10a shows a cross-sectional view along a y-z plane of the laboratory shaker 1 in the state shown in FIG. 1b. FIG. 10b shows a cross-sectional view along a y-z plane of the laboratory shaker 1 in the state shown in FIG. 1d. FIG. 10c shows a cross-sectional view along a y-z plane of the laboratory shaker 1 in the state of FIG. 1f, without sample vessels 99.

FIG. 11a shows a section of a carrier device and a handle section with connecting device 150 of a securing mechanism according to a further embodiment of the laboratory shaker according to the invention, in connected position V1 of the connecting element, first position P1 of the pull-out and first handle position S1 of the handle.

The components relevant for connecting the carrier device 12 and the pull-out base 11 and securing them in the first position P1 are assigned to the connecting device 150. The connecting device 150 includes the closing lever 152, which serves as the connecting element 152 and is pivotably arranged on the carrier device 12-offset to the pivot axis XX1 of the handle 140-and the stop section 54, which interacts with the closing lever in the connected position V1 and is integrated on the pull-out base 11.

The connecting device 150 also works in principle without the toggle lever arrangement or the locking lever 155 described below, if the connecting position V1 and thus the first position P1 of the carrier device 12 on the pull-out base 11 is secured by the locking of the locking device 30, which is already latched in the first handle position S1 and provides for the locking of the locking element 31 at the locking opening 32, which can be released by means of actuating element 42. This will be explained briefly below.

In the embodiment example of FIGS. 11a to 11d, the connecting device 150 operates as a toggle lever arrangement, which achieves the securing of the connection position V1 by using the locking lever 155. The securing is achieved by using the dead center position of the toggle lever arrangement, which comprises a similar effect to the dead center spring of the connecting device 50, whereby the toggle lever arrangement of the connecting device 150 does not require a spring element.

The connecting device 150 here includes the locking lever 155, which is connected at its first end to the handle base 141 of the handle 140 so as to pivot about a pivot axis XX3. The other end of the locking lever 155 is pivotably connected to the firing lever at a pivot axis XX4, which is arranged at a distance from the pivot axis XX2 of the closing lever on the closing lever 152. The handle base 141, which is firmly connected to the handle 140, comprises a stop edge 142, which runs approximately horizontally and against which a side edge 155′ of the locking lever 155 abuts in the connected position V1 of the locking lever 152.

The additional securing of the connected position V1 of the locking lever 152 is achieved by a force acting on the locking lever 152 in the stop section 152a, against the pull-out direction A (which could in principle occur when an attempt is made to pull out the carrier device in position V1), causing the locking lever 155 or its side edge 155′ (upwards in the figure) to be pressed against the stop edge 142 of the handle base 141, so that any further opening movement of the pull-out or the carrier device 12 is impossible. This is also illustrated by the arrangement of the swivel axes shown in FIG. 11d.

FIG. 11d shows the arrangement of the swivel axes shown in FIG. 11a, including the swivel axis XX2 of the connecting element, the swivel axis XX3 of the locking lever and the swivel axis XX4 of the coupling point between the connecting element and the locking lever, as well as the swivel axis XX1 of the handle on the carrier device. While the pivot axes XX1 of the handle and XX2 of the locking lever are fixed to the carrier device 12, the pivot axes XX3 of the locking lever and XX4 of the coupling point are movable relative to the carrier device. All swivel axes run parallel to each other, parallel to an x-axis of a Cartesian coordinate system. In the position according to FIG. 11a, which is shown in FIG. 11d, the swivel axis XX4 is not located on a line L, which connects the centers of the swivel axes XX1 and XX3, but slightly above it. The positioning of XX4 slightly above the line L results in a force acting against the pull-out direction causing a small component “upwards” (in the positive z-direction) for XX4, but no component downwards, so that as a result the side edge of the locking lever 155 is pressed against the stop edge 142 of the handle base, and is not pressed downwards. (If the pivot axes XX4, XX3 and XX1 lie on a common line L, this would be the dead center position in which neither an upward nor a downward force component would act as a result of a force directed against the pull-out direction).

If the stop edge 142 were not present, the force component pointing upwards in the above thought experiment would generate a torque of the handle in the opposite direction to its pivoting direction required for opening, as the axis XX3 would be pushed away from the axis XX2. This would be a possible, but not further described, embodiment. Since the handle is preferably locked to the carrier device by means of locking device 30, the locking element 31 would be subjected to a lateral force due to the handle torque, but would ultimately remain locked, which counteracts any further pull-out via the locking lever 152. The stop edge 142 therefore relieves the securing mechanism 30.

The kinematics can also be determined by comparing the component and swivel axis positions in FIG. 11b when the closing lever is in the unconnected position V2.

In principle, it would also be possible to dispense with the locking lever 155, to firmly connect the locking lever 152 to the handle section 141 in the relative position shown in FIG. 11a and to dispense with the connection of the locking lever to the carrier device, which is carried out via XX2 in FIG. 11a. Opening of the connected position V1 of the locking lever or pulling out of the carrier device at the base via the stop 154 is then prevented, whereby the forces occurring in the process are transmitted directly to the locking device 30 of the handle position S1. The locking device 30 would then have to be configured to be correspondingly secure. In contrast, in the embodiment examples of FIGS. 5+6 and FIG. 11, the force of the pull-out does not act directly on the locking device 30, which can be dimensioned to be correspondingly comfortable for the user.

FIG. 11c shows a perspective view of the connecting device 150 in position V2 as shown in FIG. 11b. The closing lever 152 is enclosed or guided by the parallel side walls 141a and 141b of the handle base 141.

FIG. 12 shows a further embodiment example of the locking device 30′ of a securing mechanism of a laboratory shaker according to the invention, in a perspective lateral oblique detailed view. The locking device 30′ comprises an actuating pin 30′ which can be manually pulled out against a spring force and which can be pulled out by a second hand of a user, while the handle 40′ is pulled forwards with the other hand. The locking device 30′ shown can therefore not be operated with one hand. Pulling out the actuating pin 30′ pulls a latch (not shown) integrally connected to the actuating pin out of a latch opening (not shown) provided on the carrier device, so that the handle becomes pivotable.

FIG. 13 shows a sample platform with stop elements for stopping the sample platform 14 and its carrier device 12 on a pull-out base 11 of a laboratory shaker according to the invention, in a perspective view from above. Several in particular prism-shaped stop elements 14a, which are firmly connected to the underside of the sample platform 14 (but which could alternatively also be firmly connected to the carrier device 12), are arranged so that in the first position P1 of the pull-out they strike against complementarily shaped stop elements 11a, which are arranged on the upper side of a sub-platform of the base 11. These are shown in FIG. 14.

FIG. 14 shows a base with stop elements 11a for attaching the complementary stop elements 14a of the sample platform 14 of a laboratory shaker according to the invention shown in FIG. 13, in a perspective sideways oblique detailed view. The sub-platform 11 of the base is a load-bearing plate that is placed on the connecting elements of a shaking device and fastened there. It comprises several sliding blocks 17, in particular made of plastic, on which at least one sliding element, in particular a plate, of the carrier device 12 or the sample platform 14 rests when the sample platform, loaded or unloaded with samples, is moved between the first position P1 and the second position P2 by means of the pull-out.

FIG. 15a shows a side perspective bottom view of a detail of the carrier device of the laboratory shaker provided with sample platform 14″ according to a further preferred embodiment of the invention, wherein the securing mechanism is shown, with which the first position P1 of the carrier device can be secured, with the handle of the securing mechanism in the horizontal, second handle position S2, and with the relative position R2 of the guide element or the connecting element relative to the constrained guide. The basic principle of the arrangement of FIG. 15a corresponds to that of FIG. 5c. Similar components in FIGS. 5c and 15a therefore have the same or similar reference signs.

The arrangement in FIG. 15a has been modified in detail compared to that in FIG. 5c. On the one hand, these details ensure that the overall height of the already flat arrangement of base 11″, carrier device 12″ and carrier platform 14″ of FIG. 15a is further reduced compared to FIG. 5c. In particular, the vertical position of the constrained guide section 60″ has been shifted further vertically upwards in the direction of the carrier platform compared to the corresponding part 60, in particular by the hook 54″ having a position that is shifted upwards compared to the part 54. Instead of the guide pins 53, a guide section equipped with a sliding bush 53″ is provided on both sides in the extension of the coupling rod 52a″. This interacts with the special design of the recess 11b″, 11c″ of the base plate 11″. While the recess 11b of the base 11 is shaped to accommodate the constrained guide section 60 including the connecting element 52 without restriction, the two sliding bushes 53″ widen the constrained guide section 60″ in such a way that the coupling rod 52a″ is prevented from engaging in the recess 11b″ while the carrier device (with horizontal handle in S2) is moved from the position P2 into the position P1 and the sliding bushes 53″ slide along the underside of the base plate 11″, along the sliding surfaces marked in FIG. 15c along the sliding surfaces marked in FIG. 15c. Only in position P1 do the sliding bushes 53″ reach the area of the recess section 11c″, which is wider than the recess section 11b″, so that the sliding bushes 53″ engage in the recess section 11c″ and are guided upwards along the constrained guide towards the carrier platform 14″ by pivoting the lever 40 from S2 to S1.

FIG. 15b shows the view from FIG. 15a, with the handle 40 of the securing mechanism in a vertical, first handle position S1, and with the relative position R1 of the guide element 53″ or the connecting element 52″ relative to the constrained guide 60″. FIG. 15c shows, similar to FIG. 15b, the view from FIG. 15a, with the handle 40 of the securing mechanism in a vertical, first handle position S1, and with the relative position R1 of the guide element 53″ or of the connecting element 52″ relative to the constrained guide 60″.

FIG. 16a shows a base 11″ with stop elements 11a1″, 11a2″ for attaching the complementary stop elements 14a″ of a sample platform 14″ of a laboratory shaker according to the invention, in a perspective lateral oblique detailed view. The stop elements serve to fix and position the carrier platform 14″ on the base 11″ in position P1. When position P1 is reached, the carrier platform 14″, which is mounted on the carrier device 12 in the direction of the x-y plane, is pressed against the base 11″. This is done by pressing one stop element 14a″ against the stop element 14a1″ with flat stop wall 11a1_1″ and the stop element 14a2″ with V-shaped stop wall 11a2_1″. The stop surfaces form an acute angle with the base plate 11″ in such a way that an approximately wedge-shaped receiving space is created in each case, in which the corresponding wedge section of the complementary stop section 14a″ of the carrier plate can engage-see FIG. 16d. The two stop elements 11a1″, 11a2″ realize a position-tolerant stop according to the floating bearing/fixed bearing principle, whereby the stop element 14a″ is the floating bearing and the stop element 14a″ is the fixed bearing.

The two stop elements 11a1″, 11a2″ are each preferably made of metal, e.g. brass, bronze, stainless steel or ceramic, or an abrasion-resistant plastic. This reduces the risk or prevents unacceptable abrasion of the two stop elements 11a1″, 11a2″ caused by movement, in particular the shaking movement of a laboratory shaker. The plain bearings 17 are preferably made of plastic.

These measures and preferred designs of the laboratory shaker result in easy handling. In particular, the carrier platform and the pull-out mechanism can be easily removed. The carrier platform is only fixed in the last position P1, otherwise it can be easily removed. This makes the laboratory shaker easy to clean and maintain, which increases operational safety and service life.

The mechanical construction of the laboratory shaker 1 according to the embodiment examples can be described alternatively as follows:

The chassis forms the basis of the entire appliance 1. A shaking device (X-Drive) is located on the chassis. A transmission plate (not visible) sits on the X-drive. The coupling rods or fastening parts 72 are located on the transmission plate and run vertically upwards through the chamber openings 2b. An insert element 75 sits in the chamber opening. The sub-platform 11, which forms the base 11 of the pull-out mechanism 10, sits on the coupling rods 72. Sub-platform 11 and pull-out plate 12d (in one variant as a continuous plate 12d; otherwise as frame 12d′) are bolted to the full extension runners 15. The sample platform 14 can be placed on the pull-out plate 12d. In one embodiment (FIG. 16a-d), two mushroom-shaped clamping elements 14a″ are located on the underside of the sample platform 14. These “clamping mushrooms” communicate with clamping bearings 11a1″, 11a2″ on the sub-platform 11″ and engage through recesses in the pull-out plate. The force flow from the sample platform 14 to the clamping system to the clamping bearings 11a1″, 11a2″ is as follows: The sample platform 14 rests against a thrust piece. The clamping lever 40 pulls the pull-out plate with the sample platform lying on it with the eccentric clamping system into the hooks 54″, which are attached to the sub-platform 11″. The eccentric clamp comprises a compression spring 56 for tolerance compensation during clamping and to maintain the clamping force. With this spring-loaded clamping system, the pull-out plate incl. sample platform 11″ is pulled “tight & loose” into the clamping bearings. As a result, the sub-platform 11″, the pull-out plate and sample platform 14 are clamped together. This clamping force is greater than the maximum centrifugal force that occurs during operation of the laboratory shaker.

1. A laboratory shaker for shaking samples stored in sample vessels, comprising:
a temperature-adjustable chamber with a closable chamber opening,
a carrier device for carrying a sample platform on which the sample vessels is placeable,
a pull-out mechanism for pulling out the carrier device, which is arranged completely in the chamber in a first position, which passes through the chamber opening along a pull-out direction during manual pull-out and which is arranged outside the chamber in a second position,
a shaking device for shaking the samples,
a securing mechanism for securing and releasing the first position, with a locking device and a handle, which is fastened to the carrier device and is movable between a first handle position, in which the locking device is locked, and a second handle position, in which the locking device is unlocked,
wherein a displacement of the first handle position is blockable by the locking device and the locking device comprises a manually operable actuating element arranged on the handle, by the actuation of which the locking device is unlockable in the first handle position, such that the handle is movable into the second handle position,
and wherein the securing mechanism is unlockable by simultaneously actuating the actuating element and pulling the handle in the pull-out direction.

2. The laboratory shaker according to claim 1, wherein the securing mechanism comprises a connecting device which comprises a movably arranged connecting element, in particular movably connected to the handle, for establishing and releasing a detachable connection of the carrier device and a base of the pull-out mechanism in the first position,
wherein the handle is configured to move the connecting element by its movement, so that in a first handle position the detachable connection of the carrier device and the base of the pull-out mechanism is established and in a second handle position is released.

3. The laboratory shaker according to claim 1, wherein the connecting device comprises at least one spring element which is preferably arranged on the handle and can be tensioned in such a way that when the handle is moved from the first handle position to the second handle position by manual operation of the handle, a tension generated by the at least one spring element must be overcome when the handle is moved between the first handle position and the second handle position.

4. The laboratory shaker according to claim 3, wherein the at least one spring element is arranged between the connecting element and the handle in such a way that in the first handle position the connection of the carrier device and the base is secured by the tension of the at least one spring element.

5. The laboratory shaker according to claim 3, wherein the at least one spring element is arranged between the connecting element and the handle in such a way that a dead center position of the handle is established by means of the spring element, which must be overcome in particular by manual operation of the handle when the latter is moved between the first handle position and the second handle position,
wherein the handle can preferably be secured against pivoting into the second handle position by the tension of the at least one spring element by providing a dead center handle position lying between the first handle position and second handle position, in which in particular the dead center spring is under maximum tension.

6. The laboratory shaker according to claim 1, wherein the locking device can be locked in the second handle position, so that a displacement of the second handle position can be blocked by the locking device, and wherein the locking device can be unlocked in the second handle position by manual actuation of the actuating element, so that the handle can be moved into the first handle position.

7. The laboratory shaker according to claim 1, wherein the handle is pivotable about a first pivot axis, which is in particular localized on the carrier device, and wherein the connecting element is pivotable about a second pivot axis, which is in particular localized on the handle, and which is arranged genuinely parallel to the first pivot axis on the handle.

8. The laboratory shaker according to claim 2, wherein a constrained guide section with a constrained guide is provided on the carrier device and the connecting element comprises a guide element which can be guided by the constrained guide in the first position of the carrier device, wherein the guide element can be arranged in a first position relative to the constrained guide, in which the connection of the carrier element and a base of the pull-out mechanism is established, and can be arranged in a second position relative to the constrained guide, in which the connection of the carrier element and the base of the pullout mechanism is released, wherein the guide element is guided by the constrained guide from the first relative position into the second relative position when the handle is moved manually from the first handle position into the second handle position.

9. The laboratory shaker according to claim 8, in that the constrained guide is arranged such that the movement of the guide element in the constrained guide extends in a plane to which a pivot axis of the handle extends perpendicularly, and wherein the guide element in the first relative position is arranged in an end section of the constrained guide extending along a y-direction, and wherein the constrained guide is shaped for this purpose, guiding the guide element in a negative z-direction when the handle is moved from the first handle position into the second handle position, in particular by the constrained guide extending obliquely downwards in the z-direction starting from the end section extending along the y-direction, whereby in particular the connecting element is released from an abutment element, in particular a hook element.

10. The laboratory shaker according to claim 2, wherein an abutment element is fixedly connected to the base of the pull-out mechanism, in particular a hook element, on which the connecting element is supported in the first handle position in order to form the connection of the carrier device to the base of the pull-out mechanism and to prevent the translational relative movement of carrier device and base.

11. The laboratory shaker according to claim 10, wherein the guide element in the first gripping position is supported on the abutment element, in particular by a positive connection suitable for force transmission to form the connection of the carrier device to the base of the pull-out mechanism.

12. The laboratory shaker according to claim 1, wherein the pull-out mechanism comprises a base and wherein at least one fastening part is provided to which the base is fastened, and wherein the chamber comprises a chamber bottom with at least one opening, wherein a fastening part is arranged within an opening in each case and is connected to the shaking device, so that an oscillating movement of the fastening part parallel to the chamber bottom is enabled.

13. The laboratory shaker according to claim 12, wherein the carrier device, the pull-out mechanism and the securing mechanism are carried, preferably exclusively, by the at least one fastening part and in particular are not supported on the chamber.

14. The laboratory shaker according to claim 1, comprising the sample platform, wherein the handle comprises at least one retaining section, in particular a first bearing section, and wherein the securing mechanism comprises at least one first stopper section, in particular an abutment section, which is firmly connected to the base,
wherein the carrier device is secured in the first position of the carrier device and in the first gripping position of the handle by positioning the sample platform on the at least one positioning element and holding it between the at least one retaining section of the handle and the at least one first stopper section of the base.

15. The laboratory shaker according to claim 1, wherein the chamber comprises a chamber base and wherein the pull-out mechanism comprises a base and at least one pull-out element, which in particular forms a component of a rail system of the pull-out mechanism and which is arranged movably on the base along the pull-out direction, and wherein the carrier device is fastened to at least one pull-out element,
wherein the laboratory shaker comprises a fastening device which comprises at least one fastening part, in particular arranged on the chamber base and connected to the shaking device, and at least one connecting part, with which the base can be detachably fastened to the at least one fastening part, wherein the at least one fastening part and the at least one connecting part are set up for manual and tool-free fastening.

16. The laboratory shaker for shaking samples stored in sample vessels, comprising:
a temperature-adjustable chamber with a chamber opening,
a carrier device for carrying a sample platform,
the sample platform for carrying sample vessels, which is arranged on the carrier device,
a pull-out mechanism fixed in the chamber for the pull-out of the carrier device, which in a first position is arranged completely in the chamber, which during manual pull-out passes through the chamber opening along a pull-out direction and which in a second position is arranged outside the chamber,
wherein the pull-out mechanism comprises a base and at least one pull-out element which is arranged movably along the pull-out direction on the base, and wherein the carrier device is fastened to the at least one pull-out element, which in particular forms a component of a rail system of the pull-out mechanism, and which comprises at least one positioning element by means of which the sample platform can be positioned on the carrier device,
a shaking device for moving the pull-out mechanism and the carrier device in the first position,
wherein the laboratory shaker comprises a securing mechanism for securing the first position of the carrier device and the securing mechanism includes a handle which is fastened to the carrier device, which is movable between a first handle position and a second handle position and which comprises at least one retaining section, in particular a first bearing section, and wherein the securing mechanism comprises at least one first stopper section, in particular abutment section, which is firmly connected to the base,
wherein the carrier device is secured in the first position of the carrier device and in the first gripping position of the handle by positioning the sample platform on the at least one positioning element and holding it between the at least one retaining section of the handle and the at least one first stopper section of the base.

17. The laboratory shaker according to claim 16, wherein the at least one retaining section of the handle is pressed against the sample platform in the first handle position by a spring element of the securing mechanism, in particular by a dead center spring, and presses the latter against the second stopper section serving at least as an abutment section via at least one second stopper section firmly connected to the carrier device.

18. The laboratory shaker according to claim 16, wherein the at least one second stopper section is configured to form a tongue-and-groove connection movable along the pull-out direction, and/or comprises a ramp-shaped course along the pull-out direction, with which the sample platform can be fixed in the first position with respect to a z-direction of the base.

19. A laboratory shaker for shaking samples stored in sample vessels, comprising:
a temperature-adjustable chamber with a chamber base and a chamber opening,
a carrier device for carrying a sample platform on which the sample vessels can be placed,
a pull-out mechanism for the pull-out of the carrier device, which in a first position is arranged completely in the chamber, which during manual pull-out passes through the chamber opening along a pull-out direction and which in a second position is arranged outside the chamber,
a shaking device for moving the pull-out mechanism and the carrier device in the first position,
wherein the pull-out mechanism comprises a base and at least one pull-out element, which in particular forms a component of a rail system of the pull-out mechanism and which is arranged movably along the pull-out direction on the base, and wherein the carrier device is fastened to the at least one pull-out element,
wherein the laboratory shaker comprises a fastening device which comprises at least one fastening part, in particular arranged on the chamber base and connected to the shaking device, and at least one connecting part, with which the base can be detachably fastened to the at least one fastening part, wherein the at least one fastening part and the at least one connecting part are set up for manual and tool-free fastening.

20. The laboratory shaker according to claim 19, wherein in each case a fastening part and a connecting part can be screwed together and comprise a fine thread, or wherein the fastening device comprises at least one quick-action clamping device for fastening the base.

21. (canceled)
